(19) United States
(12) Patent Application Publication
(10) Pub. No.: US 2018/0002439 A1
(43) Pub. Date: Jan. 4, 2018

(54) ANTI-MESOTHELIN ANTIBODIES AND USES THEREOF

(71) Applicants: Institut National de la Sante et de la Recherche Medicale (INSERM), Paris (FR); Universite d'Aix Marseille, Marseille 7 (FR); Centre National de la Recherche Scientifique (CNRS), Paris (FR); Institut Jean Paoli & Irene Calmettes, Marseille (FR); SRI International, Menlo Park, CA (US)

(72) Inventors: Daniel BATY, Marseille (FR); Patrick CHAMES, Marseille (FR); Brigitte KERFELEC, Marseille (FR); Nathalie SCHOLLER, Menlo Park, CA (US)

(21) Appl. No.: 15/635,855

(22) Filed: Jun. 28, 2017

Related U.S. Application Data
(60) Provisional application No. 62/357,185, filed on Jun. 30, 2016.

(51) Int. Cl. C07K 16/30; G01N 33/574

(52) U.S. Cl. CPC C07K 16/30; G01N 33/574; C07K 16/3069; G01N 33/57492; C07K 2317/34; C07K 2317/22; C07K 2317/14; C07K 2317/569; C07K 2317/94; G01N 2333/705; C07K 2317/92

(57) ABSTRACT

The present disclosure provides isolated nanobodies based on camelid VHH domains that specifically bind to mesothelin with high affinity. Also disclosed are conjugate constructs comprising the isolated nanobodies derivatized to allow conjugation to or that are conjugated to accessory moieties, such as biologically active moieties (e.g., a cytotoxin, a non-cytotoxic drug, a radioactive agent, a protein, or an enzyme) or detectable markers (e.g., a fluorescent label or biotin). Nucleic acid molecules encoding the nanobodies and conjugate constructs, expression vectors, host cells and methods for expressing the nanobodies and conjugate constructs are also provided. Pharmaceutical compositions comprising the nanobodies and conjugate-constructs as described herein are also provided. The present invention is also directed to methods for detecting mesothelin, as well as methods for diagnosis, treating, preventing and ameliorating mesothelin-associated diseases and conditions (e.g., a cancer characterized by altered expression of mesothelin), or a symptom thereof.

### CROSS-REFERENCE TO RELATED APPLICATIONS

This application claims priority to U.S. Provisional Application 62/357,185, filed Jun. 30, 2016, the complete contents of which are herein incorporated by reference.

### FIELD OF INVENTION

The present disclosure provides isolated nanobodies based on camelid VHH domains that specifically bind to mesothelin with high affinity. Also disclosed are conjugate constructs comprising the isolated nanobodies derivatized to allow conjugation to or that are conjugated to accessory moieties, such as biologically active moieties (e.g., a cytotoxin, a non-cytotoxic drug, a radioactive agent, a protein, or an enzyme) or detectable markers (e.g., a fluorescent label or biotin). Nucleic acid molecules encoding the nanobodies and conjugate constructs, expression vectors, host cells and methods for expressing the nanobodies and conjugate constructs are also provided. Pharmaceutical compositions comprising the nanobodies and conjugate-constructs as described herein are also provided. The present invention is also directed to methods for detecting mesothelin, as well as methods for diagnosis, treating, preventing and ameliorating mesothelin-associated diseases and conditions (e.g., a cancer characterized by altered expression of mesothelin), or a symptom thereof.

### INCORPORATION BY REFERENCE TO SEQUENCE LISTING SUBMITTED AS A TEXT FILE VIA THE OFFICE ELECTRONIC FILING SYSTEM

A Sequence Listing named “Seq_Mes.txt” including SEQ ID NO:1 through SEQ ID NO:12 (comprising the nucleic acid and/or amino acid sequences disclosed herein) has been submitted herewith in ASCII text format via EFS-Web and is incorporated herein by referenced in its entirety. Thus the sequence listing constitutes both the paper and computer readable form thereof The Sequence Listing was first created using PatentIn 3.5 on Jun. 27, 2016, and is 8 KB in size. The incorporated sequence descriptions and Sequence Listing comply with the rules governing nucleotide and/or amino acid sequence disclosures in patent applications as set forth in 37 C.F.R. §§1.821-1.825.

### BACKGROUND OF THE INVENTION

Antibodies and antibody fragments are widely used in oncology for nanotechnology-based diagnostic, therapeutic, and prognostic assays (see, e.g., Chen et la., Biosens Bioelectron 24(2009), 3399-3411; Chikkaveeraiah et al., Acs Nano 6(2012), 6546-6561; Choi et al., Sensors 10(2010), 428-455; Perfezou et al., Chem Soc Rev 41(2012), 2606-2622; Tang et al., Analyst 138(2013), 981-990). In particular, the diagnosis and therapy of ovarian cancer (OC), the fourth leading cause of cancer deaths among women in the United States despite its relatively low incidence (50 cases per 100,000 women), could benefit from the development of sensitive immunosensors and nanoparticles for targeted diagnostic and therapeutic applications. Several cancer immunotherapies are being developed to target mesothelin, a differentially expressed cancer biomarker with limited normal expression that is upregulated in a variety of epithelial tumors (see, e.g., Kelly et al., Mol Cancer Ther 11(2012), 517-525; Tchou et al., Breast Cancer Res Treat 133(2012), 799-804). The cell surface-associated form of mesothelin is highly expressed compared to normal tissues in adenocarcinomas of the ovary and pancreas and in epithelial mesotheliomas (Nomura et al., International surgery 98(2013), 164-169), while mesothelin serum levels are elevated at diagnosis in most late stage ovarian cancer patients and in most patients with malignant mesotheliomas (MM). Serum levels of mesothelin correlate with tumor size and increase during tumor progression, and the presence of mesothelin in MM pleural fluid can help to better discriminate mesothelioma from pleural metastasis (Chang et al., Proc Natl Acad Sci U S A 93(1996), 136-140; (1996); Scholler et al., Proc Natl Acad Sci U S A 96(1999), 11531-11536; Urban et al., Hematol Oncol Clin North Am 17(2003), 989-1005, ix; Ordonez, Mod Pathol 16(2003), 192-197; Robinson et al., Lancet 362(2003), 1612-1616; Hassan et al., Clin Cancer Res 12(2006), 447-453; Andersen et al., Cancer 113(2008), 484-489; Fukamachi et al., Biochem Biophys Res Commun 390(2009), 636-641). The OC fatality-to-case ratio remains exceedingly high due to a lack of accurate tools to diagnose early-stage disease when a cure is still possible. Strategies targeting mesothelin as an OC biomarker (Scholler et al., Proc Nail Acad Sci USA 96(1999), 11531-11536) using non-invasive, cost-effective tests have been developed. For instance, the Ov569 antibody demonstrated the presence of soluble forms of mesothelin in patients with ovarian or pancreatic cancers, or mesothelioma (Robinson et al., Lancet 362(2003), 1612-1616; Hassan et al., Clin Cancer Res 12 (2006), 447-453; McIntosh et al., Gynecol Oncol 95 (2004), 9-15; Rosen et al., Gynecol Oncol 99(2005), 267-277; Ho et al., Clin Cancer Res 13(2007), 1571-1575; Scholler et al., Clin Cancer Res 12(2006), 2117-2124) and permitted the development of the double determinant ELISA assay now commercialized by Fujirebio, Inc. as MESOMARK®.

Recombinant antibodies that recognize mesothelin are advantageous for developing next generation antibody-based diagnostic immunosensors or therapeutic immunotherapies because of the flexibility to incorporate various tags or functional groups for site-specific and oriented attachment of antibody fragments to surfaces. One type of recombinant antibody fragment is the single chain variable fragment (scFv), which is a genetically engineered antibody fragment that contains two electrostatically stabilized domains derived from natural IgGs. Two examples of scFvs that have been successfully used to recognize mesothelin are the SS(scFv)PE38 recombinant immunotoxin, which was isolated from an antibody phagemid library derived from mice immunized with DNA encoding mesothelin (Chowdhury et al., Proc Natl Acad Sci USA 95(1998), 669-674) and P4 (Bergan et al., Cancer Lett 255(2007), 263-274) which is a human-derived scFv identified by yeast-display scFv screening. The SS(scFv)PE38 was subsequently bioengineered to increase the scFv stability by including a disulfide bond instead of a flexible linker between the two scFv domains (Fan et al., Mol Cancer Ther 1(2002), 595-600; Kreitman et al., Clin Cancer Res 15(2009), 5274-5279; Tang et al., Anticancer Agents Med Chem 13(2013), 276-280). Mesothelin in the plasma of cancer patients can potentially interfere with immunotargeting strategies by acting as a competitive inhibitor and reducing tumor targeting. However, anti-mesothelin P4-targeted chimeric antigen receptor T cells challenged with ovarian cancer cells expressing high or low levels of mesothelin resisted functional inhibition by soluble mesothelin protein, even at supraphysiological levels, which suggests that soluble mesothelin may not compromise mesothelin-targeted therapeutic approaches (Lanitis et al., Mol Ther 20(2012), 633-643). Nevertheless, the poor stability of scFv fragments in vivo often remains problematic (Honegger, Handb Exp Pharmacol, (2008), 47-68).

Accordingly, there is a need in the art for additional agents that target and modulate the activity of mesothelin, e.g., for the diagnosis and treatment of diseases and conditions associated with mesothelin expression, e.g., the dysregulation of mesothelin expression.

### SUMMARY OF THE INVENTION

The present invention provides isolated nanobodies (also referenced herein as “Nb” or “Nbs”) that specifically bind to mesothelin with high affinity, in particular, mesothelin as expressed on the surface of a cell (e.g., a cancer cell). The nanobodies are based on the single variable domain, i.e., the VHH domain, of camelid HcAbs (camelid heavy-chain only antibodies) specific for mesothelin. The invention provides Nb-based tools that specifically recognize mesothelin for multiple biomedical applications including, but not limited to the detection and/or targeting of mesothelin for screening, diagnosis and/or treatment of a mesothelin-associated disease, disorders or conditions (e.g., cancer), or symptom thereof. The isolated nanobodies disclosed herein find use at least in part due to their inherent in vivo and in vivo stability.

Accordingly, the invention provides an isolated nanobody that binds to mesothelin. The nanobodies disclosed herein may be monoclonal and/or exhibit at least one of the following properties:
(a) binds to mesothelin with a K_{D} of at least 5×10⁻⁸ M or less;
(b) cross-competes with the nanobody having the amino acid sequence as set forth in FIG. 9A (SEQ ID NO:1) or in FIG. 9B (SEQ ID NO:2) for binding to mesothelin; and
(c) cross-competes with the nanobody expressed by a host cell comprising the nucleic acid sequence as set forth in FIG. 10A (SEQ ID NO:3) or in FIG. 10B (SEQ ID NO:4), or a degenerate variant thereof, for binding to mesothelin.

In preferred embodiments, the nanobodies of the invention exhibiting one or both of properties (b) and (c) cross-compete with the reference nanobodies for binding to membrane bound (e.g., on the surface of a cancer cell) or soluble mesothelin, wherein the mesothelin is as reported in Scholler et al., Cancer Lett. 247(2007), 130-136 (herein incorporated by reference in its entirety), i.e., the mesothelin derived from transcript variant (1) or (2) of the MSLN gene (NCBI accession number NM_005823 or accession number NM_013404, respectively); comprises the amino acid sequence set forth in FIG. 11A (SEQ ID NO:5); is encoded by the nucleic acid sequence set forth in FIG. 11B (SEQ ID NO:6); and/or is the mesothelin expressed by a host cell comprising the nucleic acid sequence SEQ ID NO:6. As used herein “cross-competes for binding” includes the binding of two nanobodies to the same epitope of mesothelin as well as the condition where the binding of one nanobody to mesothelin prevents the binding of the other nanobody although the two nanobodies may not bind the same epitope, i.e., wherein the binding of one nanobody sterically hinders the binding of the other nanobody to mesothelin.

In certain embodiments, the exemplary isolated nanobodies that specifically bind mesothelin according to the invention comprise one or more of, two or more of, or all three of (a) a VHH domain CDR1 comprising the amino acid sequence of GIDLSLYR (SEQ ID NO:7) or GSIFGIRT (SEQ ID NO:10); (b) a VHH domain CDR2 comprising the amino acid sequence of ITDDGTS (SEQ ID NO:8) or ITMDGRV (SEQ ID NO:11); and (c) a VHH domain CDR3 comprising the amino acid sequence of NAETPLSPVNY (SEQ ID NO:9) or RYSGLTSREDY (SEQ ID NO:12). The invention also pertains to isolated nanobodies that specifically bind mesothelin comprising one or more of, two or more of, or all three of (a) a VHH domain CDR1 comprising the amino acid sequence of GIDLSLYR (SEQ ID NO:7); (b) a VHH domain CDR2 comprising the amino acid sequence of ITDDGTS (SEQ ID NO:8); and (c) a VHH domain CDR3 comprising the amino acid sequence of NAETPLSPVNY (SEQ ID NO:9). Further exemplary isolated nanobodies that specifically bind mesothelin according to the invention comprise one or more of, two or more of, or all three of (a) a VHH domain CDR1 comprising the amino acid sequence of GSIFGIRT (SEQ ID NO:10); (b) a VHH domain CDR2 comprising the amino acid sequence of ITMDGRV (SEQ ID NO:11); and (c) a VHH domain CDR3 comprising the amino acid sequence of RYSGLTSREDY (SEQ ID NO:12).

In preferred embodiments, the isolated nanobodies specifically binding mesothelin as described herein comprise
(i) (a) a VHH domain CDR1 comprising the amino acid sequence of SEQ ID NO:7; (b) a VHH domain CDR2 comprising the amino acid sequence of SEQ ID NO:8; and (c) a VHH domain CDR3 comprising the amino acid sequence of SEQ ID NO:9; or
(ii) (a) a VHH domain CDR1 comprising the amino acid sequence of SEQ ID NO:10; (b) a VHH domain CDR2 comprising the amino acid sequence of SEQ ID NO:11; and (c) a VHH domain CDR3 comprising the amino acid sequence of SEQ ID NO:12.

The invention also relates to isolated nanobodies that specifically bind mesothelin, which isolated nanobodies comprise or consist of the VHH domain having the amino acid sequence

(SEQ ID NO: 1)
QVQLVQSGGGLVHPGGSLRLSCAASGIDLSLYRMRWYRQAPGKERDLV
ALITDDGTSYYEDSVKGRFTITRDNPSNKVFLQMNSLKPEDTAVYYCN
AETPLSPVNYWGQGTQVTVS;
or
(SEQ ID NO: 2)
QVQLVQSGGGLVQAGGSLRLSCAPSGSIFGIRTMDWYRQAPGKERELV
ARITMDGRVFHADSVKGRFSGSRDGASNAVYLQMNSLKPDDTAVYYCR
YSGLTSREDYWGPGTQVTVSS.

In some examples, the isolated nanobodies disclosed herein comprise or consist of an amino acid sequence that is at least 80%, at least 85%, at least 90%, at least 95%, at least 96%, at least 97%, at least 98% or at least 99% identical to SEQ ID NO:1 or SEQ ID NO:2, wherein the isolated nanobody specifically binds to mesothelin as disclosed herein. The isolated nanobodies specifically binding mesothelin and having an amino acid sequences that is at least 80%, at least 85%, at least 90%, at least 95%, at least 96%, at least 97%, at least 98% or at least 99% identical to SEQ ID NO:1 or SEQ ID NO:2 may further exhibit none, one, two or all three of the properties, (a) binding to mesothelin with a K_{D} of at least 5×10⁻⁸ M or less; (b) cross-competing with the nanobody having the amino acid sequence SEQ ID NO:1 or SEQ ID NO:2 for binding to an epitope of mesothelin; and (c) cross-competing with the nanobody expressed from a host cell comprising the nucleic acid sequence SEQ ID NO:3 or SEQ ID NO:4 for binding to an epitope of mesothelin.

Accordingly, nanobodies disclosed herein include an isolated nanobody comprising the amino acid sequence of SEQ ID NO:1 or an amino acid sequence that is at least 80% identical thereto, wherein the isolated nanobody specifically binds mesothelin as disclosed herein. Isolated nanobodies disclosed herein also include an isolated nanobody comprising the amino acid sequence of SEQ ID NO:2 or an amino acid sequence that is at least 80% identical thereto, wherein the isolated nanobody specifically binds mesothelin as disclosed herein.

In some embodiments, the nanobodies disclosed herein are humanized. Exemplary nanobodies of the invention are humanized nanobodies comprising one or more of a VHH domain CDR1 comprising the amino acid sequence of GIDLSLYR (SEQ ID NO:7), a VHH domain CDR2 comprising the amino acid sequence of ITDDGTS (SEQ ID NO:8), and a VHH domain CDR3 comprising the amino acid sequence of NAETPLSPVNY (SEQ ID NO:9); or comprising one or more of a VHH domain CDR1 comprising the amino acid sequence of GSIFGIRT (SEQ ID NO:10), a VHH domain CDR2 comprising the amino acid sequence of ITMDGRV (SEQ ID NO:11), and a VHH domain CDR3 comprising the amino acid sequence of RYSGLTSREDY (SEQ ID NO:12).

In some embodiments, the disclosed nanobodies bind mesothelin with a dissociation constant (K_{D}) of about 5×10⁻⁸ nM or less, about 45 nM or less, about 40 nM or less, about 35 nM or less, about 30 nM or less, about 25 nM or less, about 20 nM or less, or about 15 nM or less. In preferred embodiments, the dissociation constant is determined using surface plasmon resonance analysis, e.g., BlAcore analysis, according to standard methods known in the art.

Also provided herein is an isolated nucleic acid encoding any of the nanobodies specifically binding mesothelin disclosed herein. The isolated nanobody may have an amino acid sequence encoded by the nucleic acid sequence comprising

(SEQ ID NO: 3; FIG. 10A)
caggtgcagctggtgcagtctgggggaggcttggtgcaccctgggggg
tctctgagactctcctgtgcagcctctggaatcgacctcagtctttat
cgcatgcgctggtatcgccaggctccaggaaaggagcgcgacttggtc
gcacttataactgatgatggtacttcgtactatgaagactccgtgaag
ggccgattcaccatcaccagggacaatccctcgaacaaggtgtttctg
caaatgaacagcctgaaacctgaggacacggccgtctattactgtaat
gcagagacgcctttatcgccggtcaactactggggccaggggacccag
gtcactgtctcctc;
or
(SEQ ID NO: 4; FIG. 10B)
caggtgcagctggtgcagtctgggggaggattggtgcaggctgggggc
tctctgagactctcctgtgcaccctctggaagcatcttcggtatccgt
accatggactggtaccgccaggctccagggaaggagcgcgagttggtc
gcacgaattacgatggatggtcgggtattccatgcagactccgtgaag
ggccgattctccggctccagagacggcgcctcgaacgcggtgtatctg
caaatgaacagcctgaaacctgacgacacggccgtctattactgtcga
tatagtggcttaacctcaagggaggactactggggcccggggacccag
gtcaccgtctcctca.

Also encompassed by the invention are isolated nucleic acid sequences that are degenerate variants of SEQ ID NO:3 or SEQ ID NO:4, encoding amino acid sequences SEQ ID NO:1 or SEQ ID NO:2. In certain embodiments, the invention encompasses isolated nucleic acid sequences encoding a nanobody comprising (a) a VHH domain CDR1 comprising the amino acid sequence of SEQ ID NO:7, a VHH domain CDR2 comprising the amino acid sequence of SEQ ID NO:8, and a VHH domain CDR3 comprising the amino acid sequence of SEQ ID NO:9; (b) a VHH domain CDR1 comprising the amino acid sequence of SEQ ID NO:10, a VHH domain CDR2 comprising the amino acid sequence of SEQ ID NO:11, and a VHH domain CDR3 comprising the amino acid sequence of SEQ ID NO:12; (c) the amino acid sequence SEQ ID NO:1; or (d) the amino acid sequence SEQ ID NO2.

The isolated nucleic acids provided herein may or may not be operably linked to a promoter as known in the art or described herein. Also provided are expression vectors comprising the isolated nucleic acid molecules disclosed herein. Isolated host cells comprising the nucleic acid molecules or vectors as described herein are also provided by the invention. In some embodiments, the host cell is E. coli.

Methods of producing a nanobody (such as the host cell comprising a nucleic acid encoding any of the anti-mesothelin nanobodies described herein) comprising culturing the host cell so that the nanobody is produced, and/or recovering and/or isolating the nanobody from the host cell, are further provided. Accordingly, the invention further provides isolated nanobodies expressed by a host cell comprising (a) a nucleic acid encoding a nanobody having a VHH domain CDR1 comprising the amino acid sequence of SEQ ID NO:7, a VHH domain CDR2 comprising the amino acid sequence of SEQ ID NO:8, and a VHH domain CDR3 comprising the amino acid sequence of SEQ ID NO:9; (b) a nucleic acid encoding a nanobody having a VHH domain CDR1 comprising the amino acid sequence of SEQ ID NO:10, a VHH domain CDR2 comprising the amino acid sequence of SEQ ID NO:11, and a VHH domain CDR3 comprising the amino acid sequence of SEQ ID NO:12; (c) the nucleic acid sequence SEQ ID NO:3, or a degenerate variant thereof; or (d) the nucleic acid sequence SEQ ID NO:4, or a degenerate variant thereof.

This disclosure also provides a conjugate-construct, comprising a nanobody as described herein linked to an accessory moiety. As used in the context of the conjugate construct, the term “linked” may refer to the covalent linkage of the accessory moiety to the nanobody or may refer to noncovalent linkage of the accessory moiety to the nanobody. The accessory moiety may be a biologically active moiety (BAM), which exhibits one or more activity on a biological system rendering the BAM or conjugate-construct suitable for the treatment, prevention or amelioration of one or more diseases or conditions as disclosed herein, e.g., the treatment or amelioration of a mesothelin-associated disease or condition (e.g., cancer), or a symptom thereof. Alternatively, or additionally, the accessory moiety may exhibit no detectable biological activity, but may function as a signal or reporter moiety suitable to allow the detection of the accessory moiety or the conjugate-construct in vitro or in vivo, e.g., to aid in the screening or diagnosis of mesothelin expression and/or a mesothelin-associated disease. Further, the accessory moiety may also itself be a conjugating molecule, enabling additional covalent or non-covalent binding to further target molecules. Such conjugating accessory molecules may enable isolation of the conjugate-construct and/or screening and diagnostic methods, e.g., by binding to further signal or reporter molecules. Non-limiting examples of such conjugating accessory molecules include, e.g., biotin and hexahistidine tags as known in the art.

Where the accessory moiety is also a protein, peptide or polypeptide, it may be conjugated to the nanobody to form a conjugate-construct via a peptide-bond. The accessory moiety (e.g., BAM) may be chemically conjugated to the nanobody directly or may be linked to the nanobody through a linker group. As used throughout the disclosure, direct conjugation indicates the conjugation of the accessory moiety to any amino acid residue within nanobody using any chemical coupling known in the art or described herein suitable for the conjugation of the accessory moiety to an amino acid residue (e.g., an amino acid side chain) of the nanobody. Accordingly, direct coupling may result in one or more chemical groups spaced between the accessory moiety and the amino acid (e.g., amino acid side chain) of the nanobody, which groups form as a result of the coupling reaction as is known in the art.

Alternatively, as described herein, the accessory moiety may be conjugated to any amino acid residue within the nanobody indirectly, that is, via a linker group. Therefore, as used throughout this disclosure, indirect conjugation means that the accessory moiety (e.g., BAM) is conjugated to the linker group, which linker group is conjugated to an amino acid residue within the nanobody. The conjugation between the accessory moiety and the linker group and between the linker group and an amino acid residue of the nanobody may be any conjugation method and/or compound suitable for effecting such conjugation as described herein or as is otherwise known in the art. The conjugation between the accessory molecule and the nanobody, whether direct or indirect, may be via a cleavable or non-cleavable linker.

The direct or indirect conjugation of the accessory moiety may be directed to any amino acid residue within the nanobody as described herein. Thus, the accessory moiety may be directly or indirectly conjugated to an amino acid residue that is at the N or C terminus of the nanobody. Alternatively or additionally, the accessory moiety may be directly or indirectly conjugated to an internal amino acid residue of the nanobody. As used throughout this disclosure, an internal residue references an amino acid residue of the nanobody that is not at the terminus of the linear peptide chain of the nanobody. As is known in the art, conjugation methods (whether direct or indirect) may require the chemical modification of one or both sites of conjugation (e.g., modification of an amino acid residue within or at the terminus of the linker group, accessory molecule, and/or the nanobody disclosed herein). Accordingly, the present invention also encompasses chemical modification of the components of the conjugate-constructs disclosed herein (e.g., the linker group, accessory molecule, and/or the nanobody) described herein suitable to allow conjugation of said compounds and components. Where a linker group is present, such linker group may be any linker, e.g., a peptide linker, known in the art or disclosed herein suitable for linking the nanobody to the accessory moiety. Non-limiting examples of linker groups include peptide linkers, e.g., comprising one or more residues of glutamic acid, glycine, serine, cysteine and combinations thereof.

The invention also encompasses conjugate-constructs wherein the accessory moiety is directly linked to the nanobody. Where the conjugate-construct is lacking a linking group, the accessory moiety may be conjugated, e.g., chemically conjugated, directly to a residue within or at the terminus of the nanobody's amino acid sequence. Non-limiting examples of such chemical conjugation include covalent attachment to the peptide molecule at the N-terminus and/or to the N-terminal amino acid residue via an amide bond or at the C-terminus and/or C-terminal amino acid residue via an ester bond.

Where the conjugate-construct comprises a BAM, the BAM is expected to exert a therapeutically relevant activity on administration to an organism/subject or on delivery to one or more cells of an organism, whether in vitro or in vivo. In certain embodiments, such activity is relevant for the treatment, prevention or amelioration of a mesothelin-associated disease or condition (e.g., cancer) or a symptom thereof. Non-limiting examples of BAMs encompassed by the invention include cytotoxic agents and antineoplastic agents.

Compositions comprising a nanobody, or a conjugate-construct, disclosed herein and a pharmaceutically acceptable carrier are also provided. The nanobodies and/or conjugate constructs disclosed herein are useful in the diagnosis, screening, treatment, prevention and/or amelioration of diseases or conditions, or a symptom thereof, whose pathology involves mesothelin. As a non-limiting example, the nanobodies and conjugate-constructs disclosed herein may be of use in diagnosing or confirming the diagnosis of a cancer that expresses mesothelin in a subject, e.g., mesothelioma, prostate cancer, lung cancer, stomach cancer, squamous cell carcinoma, pancreatic cancer, cholangiocarcinoma, breast cancer or ovarian cancer. Accordingly, provided herein is a method of diagnosing or confirming the diagnosis of cancer in a subject by contacting a sample from the subject suspected of having cancer, or having been previously diagnosed with cancer, with a nanobody or conjugate-construct disclosed herein that binds mesothelin, and detecting binding of the nanobody or conjugate-construct to the sample. In certain embodiments, an increase in binding of the nanobody or the conjugate-construct to the sample relative to binding of the nanobody or the conjugate-construct diagnoses or confirms the diagnosis of cancer in the subject, wherein the diagnosis or confirmation of diagnosis may result in the modification of a treatment plan, e.g., the alteration of administered therapeutics and/or the administration of one or more cytotoxic and/or anti-neoplastic therapeutics. In some embodiments, the disclosed methods further include contacting a second antibody that specifically recognizes the nanobody or conjugate-construct (e.g., the reporter or detector label) with the sample, and detecting binding of the second antibody according to methods known in the art or described herein.

Further provided is a method of treating a subject diagnosed with or suspected to have a cancer that expresses a mesothelin (a mesothelin-associated cancer), e.g., by inhibiting the growth of a mesothelin-associated cancer cell such as a metastasis. The method may comprise (a) contacting the mesothelin-associated cancer cell with a nanobody and/or conjugate-construct disclosed herein; or (b) administering to the subject a nanobody or conjugate-construct disclosed herein such that the growth of the tumor cell is inhibited or such that the cancer is treated. Non-limiting examples of mesothelin associated cancers as known in the art include mesothelioma cell, pancreatic tumor cell, ovarian tumor cell, stomach tumor cell, lung tumor cell or endometrial tumor cell. In still other embodiments, the mesothelin-expressing tumor cell is from a cancer selected from the group consisting of mesothelioma, papillary serous ovarian adenocarcinoma, clear cell ovarian carcinoma, mixed Mullerian ovarian carcinoma, endometroid mucinous ovarian carcinoma, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma and breast adenocarcinoma.

### BRIEF DESCRIPTION OF THE DRAWINGS

FIG. 1: (A) Schematic illustrating the selection of anti-mesothelin nanobodies by phage display/using the principle of phage display based selection. Llamas were immunized with recombinant human mesothelin and a nanobody library was constructed (1). Phage-nanobodies were produced (2) and selected using recombinant human mesothelin (3). Non specific phage-nanobodies were discarded (4) and mesothelin-specific phage were amplified for a second round of selection (6). After each round of selection, clones were screened by ELISA using recombinant mesothelin (8). (B) Results of phage titrations are given for each round of selection.

FIG. 2: Nanobody specificity was determined by flow cytometry using OVCAR-3, Hela, SK-OV-3, and 22Rv1 cells. (A) Mesothelin expression of the cells was determined using a commercial monoclonal antibody, K1 as control. (B) Specificity of nanobodies Nb-A1 (comprising SEQ ID NO:1) and Nb-C6 (comprising SEQ ID NO:2) for these cells. The cells were incubated with secondary antibody (black line) or anti-mesothelin antibody followed by secondary antibody (gray filled histogram).

FIG. 3: Characterization of the mesothelin epitope recognized by Nb-A1 (comprising amino acid sequence SEQ ID NO:1) and Nb-C6 (comprising amino acid sequence SEQ ID NO:2). (A) Hela cells were incubated with 1:10 serial dilutions of Nb-C6 (5 μM to 0.5 pM). Irrelevant phage-Nb (•), phage-Nb A1 (▴), or phage-Nb C6 (▪) were then added at constant and non-saturating concentrations prior to detecting bound phages with a PE-conjugated anti-M13 antibody by flow cytometry. Commercial mAb K1 antibody (∘) was added at constant and non-saturating concentration and detected with a PE-conjugated goat anti-mouse IgG antibody by flow cytometry. Error bars represent the standard deviation of experiments performed in triplicate. (B) Mammalian cell culture supernatants containing Msln-Ig were immunoblotted using anti-human IgG (H+L), Bb-A1, or a commercial antibody, K1.

FIG. 4: Affinity determination of nanobodies for mesothelin as expressed on the plasma membrane of cells. Hela cells were incubated with serial dilutions of in vitro biotinylated anti-mesothelin Nb-A1 (A), Nb-C6 (B) or commercial mAb K1 antibody (•; (C)). Bound antibodies were detected by flow cytometry using PE-conjugated streptavidin. Insets are Lineweaver-Burk plots used to determine the dissociation constant. Error bars represent the standard deviation of experiments performed in triplicate.

FIG. 5: Immunofluorescence detection of mesothelin using conjugate-constructs disclosed herein (in particular, biotinylated nanobodies). Multicellular human ovarian cancer spheroids were prepared using A1847 cells and frozen in OCT (A and B) or fixed and paraffin embedded (C-F) for detection with secondary antibody alone or with Bb A1 and secondary antibody. Cells were counterstained with DAPI. Scale bar is 50 μm in all images.

FIG. 6: Immunotargeting with nanobody-based nanoparticles. Biotinylated nanobodies (Bb-A1) were shown to mediate the targeting of superparamagnetic iron oxide nanoparticles to mesothelin. Human ovarian cancer cell lines lacking mesothelin expression (panels (A) and (C)) or expressing mesothelin (panels (B) and (D)) were incubated with a commercial mAb K1 antibody (panels (A) and (B)) or fluorescently labeled SPION (panels (C) and (D)) and analyzed by flow cytometry. The fluorescence intensity from an isotype control or untargeted SPION (gray line) is near the background fluorescence. Both mAb K1 and SPION immunotargeted to mesothelin using Bb-A1 (black line) show a fluorescence increase compared to unstained cells. The gray filled histogram in (panels (A) to (D)) represents the background cellular autofluorescence of unstained cells. (E) Binding of Cys-A1 was demonstrated using flow cytometry of HeLa cells after incubation with secondary antibody (black line) or His tagged Cys-A1 followed by secondary antibody (gray filled histogram). Specificity of Cys-A1 bioconjugated to quantum dots was demonstrated with optical imaging detection of CFSE-labeled mesothelin positive cells (A1847) and mesothelin negative cells (C30) (F); and mesothelin expression on cells using Cys A1 conjugated Qdot800 (G).

FIG. 7: Stability of nanobodies of the invention. Nanobody Nb-A1 was analyzed by flow cytometry at day 0 (A) and then incubated in PBS at −20° C., 4° C., or 37° C. for 7 days prior to analysis (B). The nanobody was also incubated in 90% human serum for 7 days at 37° C. (grey line). The black line represents the fluorescence of the negative control.

FIG. 8: Nanobody binding at physiological temperature. Nanobody-A1 modified flow cytometry compensation beads were incubated with cells lacking mesothelin expression (C30) or expressing mesothelin (A1847 and Hela) for 4 hr at 37° C. prior to fixation and nuclear staining with Hoechst. (A) Fluorescence images of representative images at 10× magnification. (B) Quantitative data from all images. Error bars represent the standard deviation. (n=42 images for each cell line; ***: p<0.0001).

FIG. 9: (A) Amino acid sequence of nanobody A1 (Nb-A1; SEQ ID NO:1); (B) amino acid sequence of nanobody C6 (Nb-C6; SEQ ID NO:2). In each sequence, the residues of the CDR1 domain are indicated by “*”, those of the CDR2 domain by “+” and those of the CDR3 domain by “#”.

FIG. 10: (A) SEQ ID NO:3, an exemplary nucleic acid sequence encoding Nb-A1 as described herein; (B) SEQ ID NO:4, an exemplary nucleic acid sequence encoding Nb-C6 as described herein.

FIG. 11: (A) SEQ ID NO:5, an exemplary mesothelin peptide immunospecifically recognized by the nanobodies and conjugate constructs described herein; (B) SEQ ID NO:6, an exemplary nucleic acid sequence encoding the mesothelin peptide sequence set forth in FIG. 11A.

### DETAILED DESCRIPTION OF THE INVENTION

Antibodies are an essential tool in preclinical and clinical diagnostic assays for ELISAs, immunohistochemistry, immunofluorescence, and flow cytometry. In addition, the rapidly growing field of nanomedicine, which uses nanobiotechnology for medical applications, incorporates antibodies into nanoparticle scaffolds to achieve molecular specificity for nanooncology diagnostic and therapeutic agents. Conventional immunoglobulins G (IgG) with a molecular weight of 150 kDa are not well-suited for nanoparticle targeting purposes, since they yield very large bioconjugates which often impedes their efficiencies. Moreover, the conditions used for mAb bioconjugation often result in random mAb orientation on the nanoparticle surface; see, Pathak et al., Nano Lett 7(2007), 1839-1845. Nanobodies (Nbs), the smallest naturally occurring antibody fragments, preserve the antigen selectivity of whole antibodies, but are extremely stable, can be produced more economically, and straightforward antibody bioengineering techniques can be used to allow oriented nanoparticle conjugation, see, Sukhanova et al., Nanomedicine 8(2012), 516-525.

As detailed in this disclosure, the invention is based on nanobodies (Nbs) derived from the immunization of llamas with mesothelin, an important cancer biomarker, to enrich the normal antibody repertoire by in vivo affinity maturation prior to creating a Nb gene library that yielded Nbs with low nanomolar affinities, i.e., high affinity nanobodies. Feasibility of functionalization of the nanobodies and conjugate constructs disclosed herein is demonstrated by two exemplary site-specific functionalization approaches (i.e., site-specific biotinylation or incorporating a free cysteine residue) for bioconjugation to superparamagnetic iron oxide nanoparticles and quantum dots using the biotin/streptavidin interaction or thiol-maleimide chemistry. This demonstrated the versatility of the mesothelin targeted nanobodies, in particular as conjugate constructs, as the ability to recognize mesothelin in conventional immunophenotyping assays (e.g., flow cytometry, immunofluorescence, and western blot) and after bioconjugation was not hindered, providing single antigen-specific reagents that can be used for both conventional and nanotechnology-based diagnostic, therapeutic, and prognostic biomedical applications. Accordingly, in one embodiment the invention provides nanobodies and/or conjugate constructs described herein comprising site specific biotinylation and/or a free cysteine residue (e.g., a cysteine residue at the N- or C-terminus of the peptide chain), e.g., allowing conjugation to accessory moieties using standard techniques known in the art.

The present invention is, in particular, based on the surprising discovery and development of mesothelin-specific nanobodies based on camelid VHH domain. Camelids use both conventional antibodies and a unique class of antibodies that lack a light chain and are composed of only heavy chains, HcAbs (Hamers-Casterman, et al., Nature 363(1993), 446-448). The binding activity of these HcAbs is generated by a single variable domain named VHH, as opposed to traditional antibodies where the paratope is assembled through the association of two variable domains (VH and VL). When produced on their own, these minimal antibody fragments (13 kDa), also known interchangeably as single domain antibodies (sdAbs) or nanobodies (Nbs), are endowed with numerous properties that make them very attractive as a minimal binding unit for developing diagnostic immunosensors and therapeutic immunotherapies through antibody engineering. For example, despite their small size, reduced paratope and monovalent binding, these antibody fragments i) have affinities typical for regular monoclonal antibodies, ii) can bind small molecules and haptens, iii) show high production yields, extreme refolding capabilities and physical stability, and iv) can recognize buried cavities at antigen surfaces not accessible to regular monoclonal antibodies using a long complementarity determining region 3 (CDR3) hypervariable loop (see, e.g., Alvarez-Rueda et al., Mol Immunol 44(2007), 1680-1690; Behar et al., Protein Eng Des Sel 21(2008), 1-10; De Genst et al., Proc Natl Acad Sci U S A 103(2006), 4586-4591; Dolk et al., Appl Environ Microbiol 71(2005), 442-450; Dumoulin et al., Protein Sci 11(2002), 500-515; Spinelli et al., Biochemistry 39(2000), 1217-1222). Libraries of Nbs generated from immunized animals represent a rich source of antigen-specific, easy-to-produce, and stable antibody fragments that can be efficiently panned by phage display methods and easily fused to various tags allowing strong and oriented immobilization to various surfaces, including nanoparticles, for biomedical applications (see, e.g., Even-Desrumeaux et al., Mol Biosyst 6(2010), 2241-2248; Even-Desrumeaux et al., Mol Biosyst 8(2012), 2385-2394; Sukhanova et al., Nanomedicine 8(2012), 516-525. Nanobodies have a high homology with the VH domains of human antibodies and can be further humanized without any loss of activity.

Nanobodies are encoded by single genes and are efficiently produced according to standard methods known in the art in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyveromyces, Hansenula or Pichia) (see, e.g., U.S. Pat. No. 6,838,254).

As further detailed herein, the anti-mesothelin nanobodies (Nbs) disclosed herein were selected by phage display for specific binding to recombinant mesothelin conjugated to magnetic beads and screened by ELISA assays for binding to plastic-immobilized mesothelin. The binding characteristics of candidate Nbs were characterized by flow cytometry using mesothelin-positive HeLa cells.

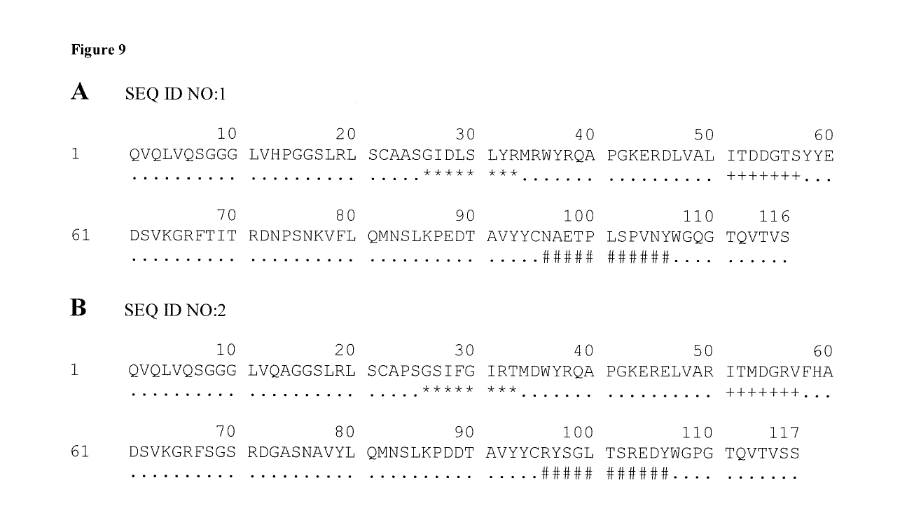

The present disclosure relates to isolated nanobodies and conjugate-constructs (i.e., comprising a nanobody covalently or non-covalently linked to an accessory moiety) that bind to membrane bound and/or soluble mesothelin, including human mesothelin. In certain embodiments, the isolated nanobodies and conjugate-constructs disclosed herein have desirable properties such as one or more of (a) binding to mesothelin with a K_{D} of at least 5×10⁻⁸ M or less; (b) cross-competing with the nanobody having the amino acid sequence SEQ ID NO:1 or SEQ ID NO:2 for binding to an epitope of mesothelin; and (c) cross-competing with the nanobody expressed from a host cell comprising the nucleic acid sequence SEQ ID NO:3 or SEQ ID NO:4 for binding to an epitope of mesothelin. In addition to the isolated nanobodies and/or conjugate constructs, the disclosure provides methods of making such nanobodies and conjugate constructs; pharmaceutical compositions containing such nanobodies and conjugate-constructs; variants or alternatives of the nanobodies such as homologous nanobodies; nanobodies with conservative modifications; and engineered and modified nanobodies, each further detailed herein below. This disclosure also provides methods of using the nanobodies and conjugate-constructs, e.g., in the diagnosis or screening of mesothelin-associated diseases or conditions (e.g., by detecting or identifying the mesothelin protein), as well as the treatment, prevention and/or amelioration of such diseases or conditions (e.g., a mesothelin expressing cancer), or a symptom thereof. The nanobodies disclosed herein can also be humanized nanobodies, derived from reference nanobodies according to standard methods known in the art. Accordingly, in certain embodiments, the invention provides humanized nanobodies comprising one or more of a VHH domain CDR1 comprising the amino acid sequence of SEQ ID NO:7, a VHH domain CDR2 comprising the amino acid sequence of SEQ ID NO:8, and a VHH domain CDR3 comprising the amino acid sequence of SEQ ID NO:9; or one or more of a VHH domain CDR1 comprising the amino acid sequence of SEQ ID NO:10, a VHH domain CDR2 comprising the amino acid sequence of SEQ ID NO:11, and a VHH domain CDR3 comprising the amino acid sequence of SEQ ID NO:12. In other embodiments, the invention provides nanobodies comprising the amino acid sequence as set forth in FIG. 9A or 9B (SEQ ID NO:1, “Nb-A1” or SEQ ID NO:2, “Nb-C6”, respectively). The invention also provides humanized versions of nanobodies comprising the amino acid sequence of SEQ ID NO:l or SEQ ID NO:2.

The invention further encompasses the modification of the disclosed nanobodies by conjugation to accessory moieties to form conjugate-constructs. A nonlimiting example of such conjugation construction is detailed in the example section, wherein an exemplary high-affinity Nb disclosed herein (comprising the amino acid sequence set forth in FIG. 9A (SEQ ID NO:1), also referenced as “Nb A1” herein) was modified to incorporate a C-terminal cysteine (Cys-Nb). Accordingly, in this embodiment, the conjugate-construct disclosed herein comprises a C-terminal cysteine as the accessory moiety. This accessory moiety is a conjugating molecule as defined herein and allows bioconjugations using thiol-maleimide chemistry. Further exemplary conjugate-constructs are also detailed in the Examples, wherein Nb A1 was modified to produce site-specific biotinylated nanobodies (Bio-Nb) by a method comprising transfer into a yeast-secreting system. The conjugate-constructs comprising cysteine (e.g., a C-terminal cysteine) or biotin (e.g., to permit further binding to streptavidin) were used to establish and validate assays using the anti-mesothelin Nb or conjugate-constructs (which may also be referenced as Nb-functionalized nanoparticles).

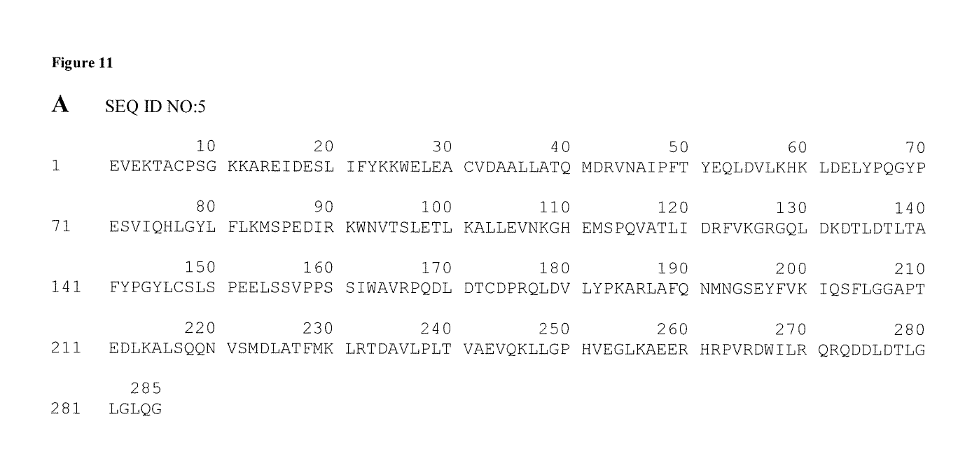

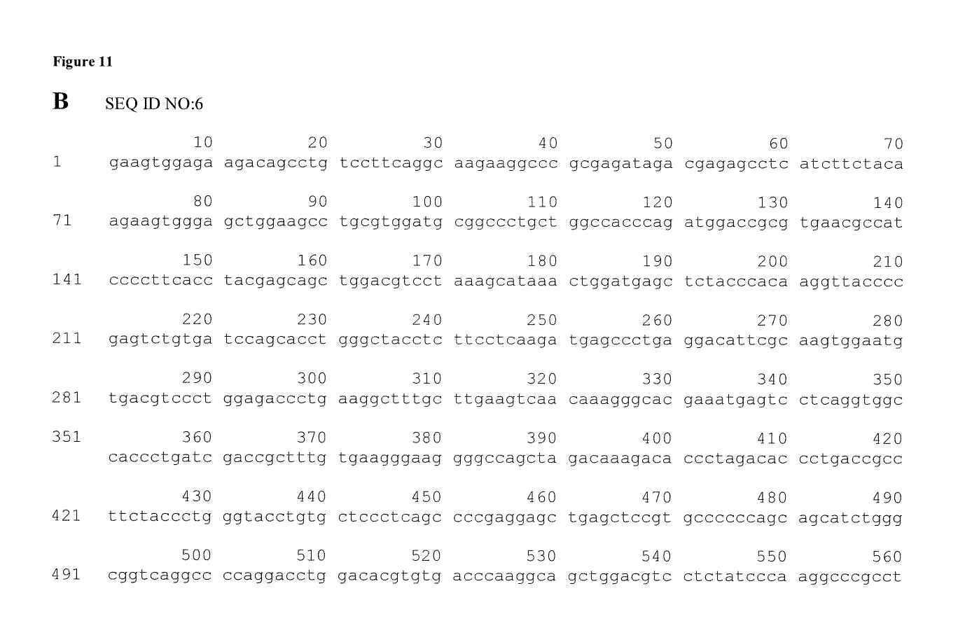

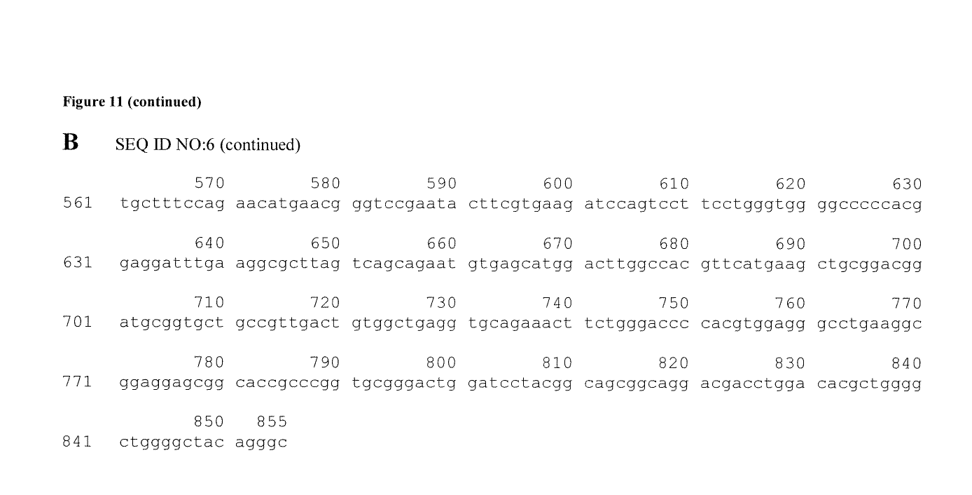

Mesothelin is a 40 kDa cell-surface glycosylphosphatidylinositol (GPI)-linked glycoprotein. The human mesothelin protein is synthesized as a 69 kD precursor which is then proteolytically processed. The 30 kD amino terminus of mesothelin is secreted and is referred to as megakaryocyte potentiating factor (Yamaguchi et al., J. Biol. Chem. 269:805 808, 1994). The 40 kD carboxyl terminus remains bound to the membrane as mature mesothelin (Chang et al., Natl. Acad. Sci. USA 93:136 140, 1996; Scholler et al., Cancer Lett 247(2007), 130-136). Exemplary nucleic acid and amino acid sequences of mesothelin provided herewith as FIGS. 11A and 11B (SEQ ID NO:6 and SEQ ID NO:5, respectively). Exemplary nucleic acid and amino acid mesothelin sequences can also be determined from the MSLN gene transcript found at (NCBI accession number NM_005823 or NCBI accession number NM_013404. Accordingly, where the nanobodies and/or the conjugate constructs disclosed herein are characterized by cross-competing with a reference antibody to mesothelin, or an epitope thereof, the mesothelin is that reported in Scholler et al., Cancer Lett 247(2007), 130-136; having the amino acid sequence SEQ ID NO:5; encoded by the exemplary nucleic acid sequence SEQ ID NO:6); and/or expressed by a host cell comprising the nucleic acid sequence SEQ ID NO:6. The competitive binding studies according to this embodiment may any assay known in the art to determine whether two antibodies or antibody-like molecules (e.g., a nanobody disclosed herein) cross-compete for binding to the same antigen, or epitope thereto, or as detailed herein.

Mesothelin also refers to mesothelin proteins or polypeptides which remain intracellular as well as secreted and/or isolated extracellular mesothelin protein, e.g., soluble mesothelin. As used herein, the term “mesothelin” also includes variants, isoforms, homologs, orthologs and paralogs. For example, nanobodies specific for mesothelin from a first species as provided herein may, in certain cases, cross-react with a mesothelin obtained from a second species. In other embodiments, the nanobodies can be specific for mesothelin obtained from only one species, e.g., human, and not exhibit cross-reactivity with mesothelin obtained from other species. Alternatively or additionally, the nanobodies specific for mesothelin obtained from a first species can cross-react with mesothelin from one or more other species but not all other species (e.g., the nanobody may specifically bind to human mesothelin and cross-react with a primate mesothelin but not cross-react with a mouse mesothelin).

As used throughout this disclosure, the phrases “a nanobody recognizing an antigen”, “a nanobody specific for an antigen”, “an antigen-specific nanobody”, and variants thereof, are used interchangeably with “a nanobody that specifically binds an antigen.”

As used herein, a nanobody or conjugate-construct that “specifically binds to mesothelin” or “specifically binds to mesothelin with high-affinity” refers to a nanobody or conjugate-construct that binds to mesothelin with a K_{D} of about 5×10⁻⁸ or less, about 40 nM, about 35 nM or less, about 30 nM, about 25 nM or less, about 20 nM or less, or about 15 nM or less. The term “does not substantially bind” or “does not significantly bind” to a indicates that the nanobody or conjugate-construct binds to a protein (e.g., in soluble form, as expressed on the surface of a cell, or as coated/attached to a substrate) with a K_{D} of about 1×10⁻⁶ M or more, 1×10⁻⁵ M or more, 1×10⁻⁴ M or more, 1×10⁻³ M or more, or 1×10⁻² M or more.

As used herein, the term “about” as characterizing an amount typically indicates a range +5% of that amount. When used in the context of a measurement or assay output, “about” indicates the value of the measurement or assay output ±the standard deviation associated with the measurement or assay as known in the art.

As used herein, the term “accessory moiety” refers to the molecule that is conjugated or linked either covalently or noncovalently to a nanobody as disclosed herein to form the conjugate-construct disclosed herein. Examples of accessory moieties include, but are not limited to, proteins (including single amino acid residues), drugs, toxins, marker molecules, detectable molecules and moieties, therapeutic agents and conjugating molecules and moieties.

The terms “conjugating”, “linking”, and variants thereof refer to the attachment of, in particular, an accessory moiety to a nanobody disclosed herein to form a conjugate-construct. The conjugation can be covalent or non-covalent. Where the nanobody and the accessory moiety are both peptides/polypeptides (including embodiments where the accessory molecule is a single amino acid residue), conjugating or linking a nanobody disclosed herein to the accessory molecule forms one contiguous polypeptide molecule from two separate molecules. The linkage can be made by chemical or by recombinant means as known in the art. For example, “chemical means” refers to a reaction between the nanobody and the accessory moiety such that there is a covalent bond formed between the two molecules to form one molecule.

As used herein, the terms “degenerate variant” and variants thereof refer to a polynucleotide encoding (a) a nanobody or conjugate-construct disclosed herein or (b) a mesothelin that includes a sequence that is degenerate as a result of the genetic code. As is well known in the art, there are 20 natural amino acids, most of which are specified by more than one codon. Therefore, the same amino acid residues and/or amino acid sequences can be encoded by multiple potential degenerate nucleotide sequences. All degenerate nucleotide sequences are included as long as the amino acid sequence of, e.g., a nanobody or conjugate-construct disclosed herein encoded by the nucleotide sequence is unchanged.

As used herein, the terms “epitope” and variants thereof refer to an antigenic determinant. As is known in the art, the antigenic determinant is formed from particular chemical groups or peptide sequences on a molecule that are antigenic, i.e. that are capable of eliciting a specific immune response, and which groups or sequences are bound by an antibody. the antigenic determinant of a protein antigen may be linear (i.e., comprising a consecutive sequence of residues within an amino acid sequence), or may be conformational (i.e., comprising residues that are not consecutive within the amino acid sequence, but that are in proximity to one another in 3-dimensional space when the protein is folded).

“Homologs” and “variants” of the nanobodies and conjugate-constructs disclosed herein are also provided. Homologs and variants of the amino acid sequences disclosed herein, e.g., of a nanobody disclosed herein that specifically binds mesothelin, are typically characterized by having at least about 80%, for example, at least about 85%, 90%, 95%, 96%, 97%, 98% or 99% sequence identity as determined over the full length alignment with the amino acid sequence of the nanobody, e.g., as determined using the NCBI Blast 2.0 and as otherwise known in the art. When less than the entire sequence is being compared for sequence identity, these fragments of the homologs/variants and the reference nanobody may possess less than 80% sequence identity. One of skill in the art will appreciate that these sequence identity ranges are provided for guidance only; it is entirely possible that strongly significant homologs could be obtained that fall outside of the ranges provided.

The term “isolated” as used herein, in particular, with regard to a nucleic acid or a peptide/polypeptide, includes a nucleic acid or peptide/polypeptide that is substantially free of other cellular or cell culture material, components, and/or chemicals. With respect to a nanobody or conjugate-construct disclosed herein, isolated indicates that the nanobody or conjugate construct is free from nanobodies or conjugate-constructs having different antigenic specificities. As is known in the art, an isolated nanobody or conjugate construct that specifically binds mesothelin may bind mesothelin from a single species and not exhibit detectable binding to the mesothelin of another species; may bind mesothelin from a number of different species but not all species of interest; or may exhibit cross-reactivity for, i.e., bind, mesothelin from all tested species

As used herein the terms “label” and variants thereof when used in the context of another protein or molecule refer to a detectable compound, composition or molecule that is conjugated directly or indirectly to a second molecule (in particular, a nanobody or conjugate-construct disclosed herein) to facilitate detection of the second molecule. Non-limiting examples of labels as known in the art include fluorescent tags, enzymatic linkages, and radioactive isotopes. In one example, a “labeled nanobody” refers to the direct or indirect conjugation of the detectable compound, composition or molecule to the nanobody. Additionally, or alternatively, the detectable compound, composition or molecule can be incorporated into the nanobody structure by means other than direct or indirect conjugation. An exemplary method of such incorporation is the replacement of an amino acid residue of the nanobody with a modified amino acid residue such that it becomes detectable (e.g., by radiolabeling). The label may be directly detectable or may be detectable only after contact with further compounds compositions or molecules. In one non-limiting example, the label may be the incorporation of a radiolabeled amino acid, which is directly detectable according to methods known in the art. In additional or alternative non-limiting examples, the label may be the attachment of biotinyl moieties to the nanobody, which are detectable following contact with marked avidin (for example, streptavidin containing a fluorescent marker or enzymatic activity that can be detected by optical or colorimetric methods as known in the art). Various methods of labeling proteins are known in the art and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ⁹⁹TC, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents (such as gadolinium chelates). In some embodiments, labels are attached by spacer arms of various lengths to reduce potential steric hindrance for the binding of the nanobody or the conjugate-construct disclosed herein.

As used herein, the terms “preventing a disease” and variants thereof refer to inhibiting the full development of a disease or a symptom thereof as evaluated by one of ordinary skill in the art, e.g., a medical practitioner. Preventing a disease may comprise therapy prior to the subject exhibiting any symptoms of the disease, or may comprise therapy after one or more symptoms are detectable, such that further development of symptoms of the disease and/or the course of the disease as is known in the art is halted. “Treating a disease” and variants thereof as used herein refers to a therapeutic intervention that reduces a sign or symptom of a disease or condition as evaluated according to standard practices in the art after the sign or symptom is detectable according to such practices. A non-limiting exemplary treatment in the context of the invention is the treatment of cancer such that the tumor burden is reduced and/or such that the number and/or size of metastases is reduced. “Ameliorating a disease” and variants thereof refer to the reduction in the number or severity of signs or symptoms of a disease, such as cancer, as evaluated according to standard methods known in the art.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The phrase “surface plasmon resonance” includes an optical phenomenon that allows for the analysis of real-time biospecific interactions by detection of alterations in protein concentrations within a biosensor matrix, for example using the BlAcore system (Pharmacia Biosensor AB, Uppsala, Sweden and Piscataway, N.J.). For further descriptions, see, e.g., Jonsson et al., Ann Biol Clin 51(1993), 19-26; Jonsson et al., Biotechniques 11(1991), 620-627; Johnsson et al., J Mol Recognit 8(1995), 125-131; and Johnnson et al., Anal Biochem 198(1991), 268-277.

The term “vector” includes a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. As used herein, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Exemplary Nanobodies

Provided herein are isolated nanobodies that specifically bind mesothelin, such as cell-surface or soluble mesothelin and, in particular, human cell surface or soluble mesothelin. The nanobodies disclosed herein may be monoclonal and/or exhibit at least one of the following properties:
(a) binds to mesothelin with a K_{D} of at least 5×10⁻⁸ M or less;
(b) cross-competes with the nanobody having the amino acid sequence SEQ ID NO:1 or SEQ ID NO:2 for binding to an epitope of mesothelin; and
(c) cross-competes with the nanobody expressed from a host cell comprising the nucleic acid sequence SEQ ID NO:3 or SEQ ID NO:4, or a degenerate variant thereof, for binding to an epitope of mesothelin.

In certain embodiments, the exemplary isolated nanobodies that specifically bind mesothelin according to the invention comprise one or more of (a) a VHH domain CDR1 comprising the amino acid sequence of GIDLSLYR (SEQ ID NO:7) or GSIFGIRT (SEQ ID NO:10); (b) a VHH domain CDR2 comprising the amino acid sequence of ITDDGTS (SEQ ID NO:8) or ITMDGRV (SEQ ID NO:11); and (c) a VHH domain CDR3 comprising the amino acid sequence of NAETPLSPVNY (SEQ ID NO:9) or RYSGLTSREDY (SEQ ID NO:12). In preferred embodiments, the isolated nanobodies specifically binding mesothelin as described herein comprise
(i) (a) a VHH domain CDR1 comprising the amino acid sequence of SEQ ID NO:7; (b) a VHH domain CDR2 comprising the amino acid sequence of SEQ ID NO:8; and (c) a VHH domain CDR3 comprising the amino acid sequence of SEQ ID NO:9; or
(ii) (a) a VHH domain CDR1 comprising the amino acid sequence of SEQ ID NO:10; (b) a VHH domain CDR2 comprising the amino acid sequence of SEQ ID NO:11; and (c) a VHH domain CDR3 comprising the amino acid sequence of SEQ ID NO:12.

Further exemplary nanobodies disclosed herein that specifically bind mesothelin include nanobodies comprising or consisting of the VHH domain having the amino acid sequence SEQ ID NO:1 or SEQ ID NO:2. In some examples, the isolated nanobodies disclosed herein comprise or consist of an amino acid sequence that is at least 80%, at least 85%, at least 90%, at least 95%, at least 96%, at least 97%, at least 98% or at least 99% identical to SEQ ID NO:1 or SEQ ID NO:2, wherein the isolated nanobody specifically binds to mesothelin as disclosed herein. The isolated nanobodies specifically binding mesothelin and having an amino acid sequences that is at least 80%, at least 85%, at least 90%, at least 95%, at least 96%, at least 97%, at least 98% or at least 99% identical to SEQ ID NO:1 or SEQ ID NO:2 may further exhibit none, one, two or all three of the properties, (a) binding to mesothelin with a K_{D} of at least 5×10⁻⁸ M or less; (b) cross-competing with the nanobody having the amino acid sequence SEQ ID NO:1 or SEQ ID NO:2 for binding to an epitope of mesothelin; and (c) cross-competing with the nanobody expressed from a host cell comprising the nucleic acid sequence SEQ ID NO:3 or SEQ ID NO:4 for binding to an epitope of mesothelin.

Accordingly, nanobodies disclosed herein include an isolated nanobody comprising the amino acid sequence of SEQ ID NO:1 or an amino acid sequence that is at least 80%, at least 85%, at least 90%, or at least 95% identical thereto, wherein the isolated nanobody specifically binds mesothelin as disclosed herein. Isolated nanobodies disclosed herein also include an isolated nanobody comprising the amino acid sequence of SEQ ID NO:2 or an amino acid sequence that is at least 80%, at least 85%, at least 90%, or at least 95% identical thereto, wherein the isolated nanobody specifically binds mesothelin as disclosed herein.

### Homologous Variants

In certain embodiments, a nanobody or conjugate construct disclosed herein comprises an amino acid sequence that is homologous to a preferred amino acid sequences as disclosed herein (e.g., SEQ ID NO:1; SEQ ID NO:2; or an amino acid sequence comprising one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; and (c) a VHH domain CDR3 comprising the amino acid sequence of SEQ ID NO:9 or SEQ ID NO:12) and wherein the nanobodies conjugate-constructs retain the desired functional properties of the anti-mesothelin nanobodies or conjugate-constructs as disclosed herein.

For example, provided herein are isolated nanobodies comprising an amino acid sequence that is at least 80% homologous to an amino acid sequence selected from the group consisting of SEQ ID NO:1; SEQ ID NO:2; or an amino acid sequence comprising one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; and (c) a VHH domain CDR3 comprising the amino acid sequence of SEQ ID NO:9 or SEQ ID NO:12, wherein isolated nanobody specifically binds to human mesothelin. A nanobody having high (i.e., 80% or greater) homology to the preferred amino acid sequences as set forth above can be obtained by mutagenesis according to any method known in the art or disclosed herein (e.g., site-directed or PCR-mediated mutagenesis of nucleic acid molecules encoding, e.g., SEQ ID NO:1 or SEQ ID NO:2), followed by testing of the encoded altered nanobody for retention of one or more desired features/properties as set forth above, e.g., using a functional assay as known in the art or described herein.

The percent homology between two amino acid sequences is equivalent to the percent identity between the two sequences. The percent identity between the two sequences is a function of the number of identical positions shared by the sequences (i.e., % homology=(number of identical positions)/(total number of positions)×100), taking into account the number of gaps, and the length of each gap, which need to be introduced for optimal alignment of the two sequences as known in the art.

A non-limiting example of a method by which the homology or % identity between two sequences can be determined is the algorithm of E. Meyers and W. Miller (Comput Appl Biosci, 4(1988), 11-17) which has been incorporated into the ALIGN program (version 2.0), using a PAM120 weight residue table, a gap length penalty of 12 and a gap penalty of 4. In addition, the percent identity between two amino acid sequences can also be determined using the Needleman and Wunsch (J Mol Biol 48(1970):444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

In certain embodiments, homologous variants of the nanobodies or the conjugate-constructs disclosed herein also encompass amino acid variants having conservative residue substitutions, i.e., nanobody amino acid sequences SEQ ID NO:1; SEQ ID NO:2; or an amino acid sequence comprising one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; and (c) a VHH domain CDR3 comprising the amino acid sequence of SEQ ID NO:9 or SEQ ID NO:12, comprising one or more conservative modifications, wherein the nanobodies or conjugate-constructs retain the desired functional properties of the nanobodies and conjugate-constructs of this disclosure. As is well known in the art, certain conservative sequence modifications can be made that do not impact antigen binding. Accordingly, the invention provides an isolated nanobody or conjugate construct comprising an amino acid sequence selected from the group consisting of SEQ ID NO:1; SEQ ID NO:2; or an amino acid sequence comprising one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; (c) a VHH domain CDR3 comprising the amino acid sequence of SEQ ID NO:9 or SEQ ID NO:12, and (d) conservative modifications thereof, wherein the nanobody specifically binds human mesothelin.

The term “conservative sequence modifications” refers to amino acid modifications that do not significantly affect or alter the binding characteristics of the nanobody or conjugate-construct containing the amino acid sequence. Such conservative modifications include amino acid substitutions, additions and deletions. Modifications can be introduced into an amino acid sequence by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of a nanobody or a conjugate-construct of this disclosure can be replaced with other amino acid residues from the same side chain family and the altered nanobody or conjugate-construct can be tested for retained function using the functional assays described herein.

### Nanobodies that Cross-Compete for Binding to Mesothelin

Also disclosed are nanobodies and conjugate-constructs that cross compete for binding to mesothelin with any of the anti-mesothelin nanobodies disclosed herein. Any competition assay known in the art or as described herein can be used to identify a nanobody or conjugate construct that competes with any of the nanobodies or conjugate-constructs described herein for binding to mesothelin. In certain embodiments, such a competing nanobody or conjugate-construct binds to the same epitope (e.g., a linear or a conformational epitope) that is bound by a nanobody or conjugate-construct described herein. Exemplary methods for a competition assay is provided in the Examples and are known in the art. Methods for mapping the epitope to which an antibody or antibody-like molecule, e.g., a nanobody or conjugate-construct disclosed herein) binds are also known in the art, see, e.g., Morris, Epitope Mapping Protocols, in Methods in Molecular Biology vol. 66 (1996, Humana Press, Totowa, N.J.).

In a non-limiting, exemplary competition assay, immobilized mesothelin is incubated in a solution comprising a first labeled nanobody or conjugate-construct that binds to mesothelin (e.g., as described herein) and a second unlabeled nanobody or conjugate-construct that is being tested for its ability to compete with the first nanobody or conjugate-construct for binding to mesothelin. As a control, immobilized mesothelin is incubated in a solution comprising the first labeled nanobody or conjugate-construct but not the second unlabeled nanobody or conjugate-construct. After incubation under conditions permissive for binding of the first nanobody or conjugate-construct to mesothelin, excess unbound nanobody or conjugate-construct is removed, and the amount of label associated with immobilized mesothelin is measured. If the amount of label associated with immobilized mesothelin is substantially reduced in the test sample relative to the control sample, then that indicates that the second nanobody or conjugate-construct competes with the first (or reference) nanobody or conjugate-construct for binding to mesothelin; see, e.g.,. Harlow and Lane (1988) Antibodies: A Laboratory Manual ch. 14 (Cold Spring Harbor Laboratory, Cold Spring Harbor, N.Y.).

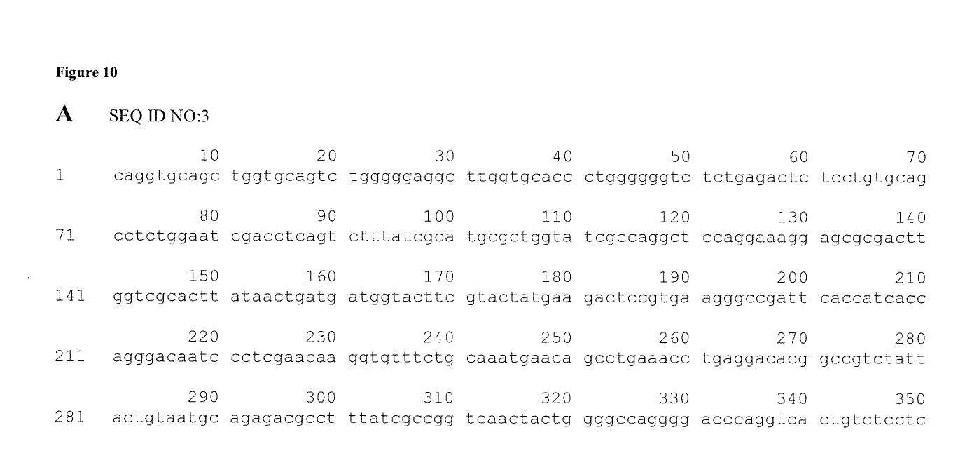

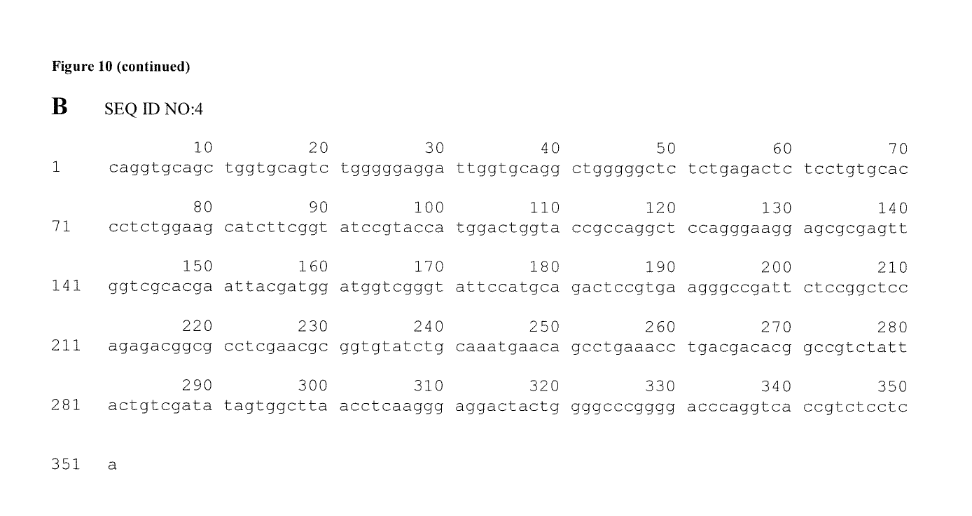

In preferred embodiments, the reference nanobody (i.e., the first nanobody or conjugate-construct as described immediately above) for cross-competition assays (i.e., the first nanobody or conjugate-construct as described immediately above) can be (a) a nanobody having the amino acid sequence as set forth in FIG. 9A (SEQ ID NO:1; Nb-A1); (b) a nanobody having the amino acid sequence as set forth in FIG. 9B (SEQ ID NO:2; Nb-C6); (c) an amino acid sequence comprising one or more of (i) a VHH domain CDR1 comprising the amino acid sequence of SEQ ID NO:7 or SEQ ID NO:10; (ii) a VHH domain CDR2 comprising the amino acid sequence of SEQ ID NO:8 or SEQ ID NO:11; and (iii) a VHH domain CDR3 comprising the amino acid sequence of SEQ ID NO:9 or SEQ ID NO:12; (d) a nanobody encoded by the nucleic acid sequence as set forth in FIG. 10A or 10B (SEQ ID NO:3 or SEQ ID NO:4, respectively); or (e) a nanobody expressed by a host cell comprising the nucleic acid sequence SEQ ID NO:3 or SEQ ID NO:4. The mesothelin used for the cross-competition assays is preferably (a) as reported in Scholler et al., Cancer Lett. 247(2007), 130-136 (herein incorporated by reference in its entirety), i.e., the mesothelin derived from transcript variant (1) or (2) of the MSLN gene (NCBI accession number NM_005823 or accession number NM_013404, respectively); (b) comprises the amino acid sequence as set forth in FIG. 11A (SEQ ID NO:5); (c) is encoded by the nucleic acid sequence as set forth in 11B (SEQ ID NO:6); or (d) is the mesothelin expressed from a host cell comprising the nucleic acid sequence SEQ ID NO:6. Such cross-competing nanobodies or conjugate-constructs can be identified based on their ability to cross-compete with a reference nanobody, e.g., Nb-A1 or Nb-C6, in standard mesothelin binding assays, including but not limited to ELISA and BlAcore analysis.

### Nucleic Acid Molecules Encoding Nanobodies and Conjugate-Constructs of the Invention

The invention also pertains to nucleic acid molecules that encode the nanobodies and/or peptide conjugate-constructs disclosed herein. The nucleic acids may be present in whole cells, in a cell lysate, in a partially purified form, or in substantially pure form, i.e., isolated. A nucleic acid is “isolated” or “rendered substantially pure” when purified away from other cellular components or other contaminants, e.g., other cellular nucleic acids or proteins, by any method known in the art and/or described herein. Non-limiting examples of techniques for purification and/or isolation of nucleic acids include alkaline/SDS treatment, CsC1 banding, column chromatography, agarose gel electrophoresis and others well known in the art (see, e.g., Ausubel, et al., (ed.), Current Protocols in Molecular Biology, Greene Publishing and Wiley Interscience, (1987) New York). A nucleic acid can be, for example, DNA or RNA and may or may not contain intronic sequences. In a preferred embodiment, the nucleic acid is a cDNA molecule.

Preferred nucleic acids molecules are those encoding amino acid sequences SEQ ID NO:1 and SEQ ID NO:2, or homologous derivatives thereof. Exemplary nucleic acids include SEQ ID NO:3 and SEQ ID NO:4, which encode SEQ ID NO:1 and SEQ ID NO:2, respectively. Also encompassed are isolated nucleic acid sequences that are degenerate variants of SEQ ID NO:3 or SEQ IDNO:4, wherein the variants encode the amino acid sequences SEQ ID NO:1 or SEQ ID NO:2, respectively.

### Recombinant Methods

The nanobodies and conjugate-constructs disclosed herein may be produced using any recombinant method and composition known in the art and/or as described herein, e.g., as described in U.S. Pat. No. 4,816,567. In one embodiment, an isolated nucleic acid encoding an anti-mesothelin nanobody or conjugate-construct is provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a nucleic acid encoding a nanobody or conjugate-construct as disclosed herein (e.g., having the amino acid sequence of SEQ ID NO:1; SEQ ID NO:2; or an amino acid sequence comprising one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; and (c) a VHH domain CDR3 comprising the amino acid sequence of SEQ ID NO:9 or SEQ ID NO:12); or (2) a vector comprising the nucleic acid of (1). The host cell may be prokaryotic or eukaryotic, including but not limited to any suitable E coli strain as known in the art or described herein (e.g., BL21DE3), a Chinese Hamster Ovary (CHO) cell or lymphoid cell (e.g., Y0, NS0, Sp20 cell). In one embodiment, a method of making a nanobody or conjugate construct that specifically binds mesothelin is provided, wherein the method comprises culturing a host cell comprising a nucleic acid encoding the nanobody or conjugate-construct, as provided above, under conditions suitable for expression of the nanobody or conjugate-construct, and optionally recovering the nanobody or conjugate-construct from the host cell (or host cell culture medium).

For recombinant production of a nanobody or conjugate-construct that specifically binds to mesothelin, a nucleic acid encoding such a nanobody or conjugate-construct, e.g, as described above, may be isolated and inserted into one or more vectors for further cloning and/or expression in a host cell. Such nucleic acid may be readily isolated and sequenced using conventional procedures.

The nucleic acids encoding the nanobodies and conjugate-constructs are typically inserted into expression vectors such that the genes are operatively linked to transcriptional and translational control sequences. In this context, the term “operatively linked” is intended to mean that a gene is ligated into a vector such that transcriptional and translational control sequences within the vector serve their intended function of regulating the transcription and translation of the gene to be expressed. The expression vector and expression control sequences are chosen to be compatible with the expression host cell used. The desired genes may be inserted into the expression vector by standard methods. Additionally or alternatively, the recombinant expression vector can encode a signal peptide that facilitates secretion of the nanobody or conjugate-construct from a host cell. The gene encoding the nanobody or conjugate-construct can be cloned into the vector such that the signal peptide is linked in-frame to the amino terminus of the nanobody or conjugate-construct gene. The signal peptide can be an immunoglobulin signal peptide or a heterologous signal peptide (i.e., a signal peptide from a non-immunoglobulin protein).

The recombinant expression vectors as disclosed herein may additionally carry regulatory sequences that control the expression of the nanobody or conjugate-construct genes in a host cell. The term “regulatory sequence” is intended to include promoters, enhancers and other expression control elements (e.g., polyadenylation signals) that control the transcription or translation of the desired genes. It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as those derived from cytomegalovirus (CMV), Simian Virus 40 (SV40), adenovirus, and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1, may be used.

For expression, the recombinant vectors as disclosed herein are transfected into a host cell. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Suitable host cells for cloning or expression of nanobody or conjugate-construct-encoding vectors include any prokaryotic or eukaryotic cells as known in the art or described herein. For example, nanobodies or conjugate-constructs disclosed herein may be produced in bacteria as further described in the Examples, see also, e.g., U.S. Pat. Nos. 5,648,237; 5,789,199; and 5,840,523; Charlton, Methods in Molecular Biology, Vol. 248 (B.K.C. Lo, ed., Humana Press, Totowa, N.J., 2003), pp. 245-254). After expression, the nanobody or conjugate-construct may be isolated from the bacterial cell paste in a soluble fraction and can be further purified.

In addition to prokaryotes, eukaryotic microbes such as filamentous fungi or yeast are also suitable cloning or expression hosts for vectors encoding the nanobodies and conjugate-constructs. Of particular interest may be fungi and yeast strains having glycosylation pathways that have been “humanized,” resulting in the production of an biomolecules, e.g., a nanobody disclosed herein, partially or fully human glycosylation pattern, see, e.g., Gerngross, Nat Biotech 22(2004), 1409-1414; Li et al., Nat Biotech 24(2006), 210-215.

Suitable host cells for the expression of the nanobodies and conjugate-constructs disclosed herein may also derived from multicellular organisms (invertebrates and vertebrates). Examples of invertebrate cells include plant and insect cells. Numerous baculoviral strains have also been identified which may be used in conjunction with insect cells, particularly for transfection of Spodoptera frugiperda cells.

Plant cell cultures can also be utilized as hosts and are well known for expressing antibodies and antibody fragments, see, e.g., U.S. Pat. Nos. 5,959,177; 6,040,498; 6,420,548; 7,125,978; and 6,417,429 (describing PLANTIBODIES™ technology for producing antibodies in transgenic plants).

Vertebrate cells may also be used as hosts, in particular, mammalian cells. Non-limiting examples of mammalian host cell lines include monkey kidney CV1 line transformed by SV40 (COS-7); human embryonic kidney line (293 or 293 cells); baby hamster kidney cells (BHK); mouse sertoli cells (TM4 cells); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells; MRC 5 cells; FS4 cells; Chinese hamster ovary (CHO) cells; and myeloma cell lines such as Y0, NS0 and Sp2/0; see also, e.g., Yazaki and Wu, Methods in Molecular Biology, Vol. 248 (B.K.C. Lo, ed., Humana Press, Totowa, N.J.), pp. 255-268 (2003).

### Methods of Engineering Peptide Sequences

The invention also encompasses nanobodies and conjugate-constructs derived from the nanobodies and conjugate-constructs disclosed herein, created by modifying their amino acid sequences and/or conjugating accessory moieties thereto. Accordingly, the structural features of a known nanobody specific for mesothelin, e.g., Nb-A1 comprising SEQ ID NO:1 or Nb-C6 comprising SEQ ID NO:2, may be used to create structurally related nanobodies that specifically bind mesothelin and that retain at least one further functional property of the nanobodies disclosed herein, i.e., retain one or more of (a) binding to mesothelin with a K_{D} of at least 5×10⁻⁸ M or less; (b) cross-competing with the nanobody having the amino acid sequence SEQ ID NO:1 or SEQ ID NO:2 for binding to an epitope of mesothelin; and (c) cross-competing with the nanobody expressed from a host cell comprising the nucleic acid sequence SEQ ID NO:3 or SEQ ID NO:4 for binding to an epitope of mesothelin. For example, one or more CDR regions of known anti-mesothelin nanobodies, can be combined recombinantly with known VHH framework regions and/or other known nanobody CDRs to create additional, recombinantly-engineered, nanobodies or conjugate-constructs specific for mesothelin, as discussed above. To create the engineered nanobody or conjugate-construct, it is not necessary to actually prepare (i.e., express as a protein) the nanobody or conjugate-construct. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, a method for preparing a nanobody or conjugate-construct specific for mesothelin is provided comprising
(a) providing a nanobody amino acid sequence of SEQ ID NO:1; SEQ ID NO:2; or an amino acid sequence comprising one or more of (i) a VHH domain CDR1 comprising the amino acid sequence of SEQ ID NO:7 or SEQ ID NO:10; (ii) a VHH domain CDR2 comprising the amino acid sequence of SEQ ID NO:8 or SEQ ID NO:11; and (ii) a VHH domain CDR3 comprising the amino acid sequence of SEQ ID NO:9 or SEQ ID NO:12;
(b) altering at least one amino acid residue within the amino acid sequence to create at least one altered nanobody sequence; and (c) expressing the altered nanobody sequence as a protein.

Standard molecular biology techniques can be used to prepare and express the altered nanobody sequence.

In certain embodiments, mutations can be introduced randomly or selectively along all or part of a coding sequence for a nanobody specific for mesothelin and the resulting modified nanobodies can be screened for binding activity and/or other functional properties as described herein. Such mutational methods are well known in the art.

Preferably, the nanobodies or conjugate-constructs disclosed herein are monoclonal, and, may or may not be humanized.

Also disclosed are cysteine engineered nanobodies and conjugate-constructs, e.g., thio-derivatives, in which one or more residues of a nanobody or conjugate-construct are substituted with cysteine residues. In particular embodiments, the substituted residues occur at accessible sites of the nanobody or conjugate-construct, and are preferably at the N- or C-terminus of the amino acid sequence. By substituting residues with cysteine, reactive thiol groups are positioned at accessible sites and may be used to conjugate the nanobody or conjugate-construct to other moieties, such as drug moieties or linker-drug moieties.

Also disclosed are conjugate-constructs, wherein a nanobody as described herein is conjugated/linked (directly or indirectly (i.e., through the use of a linker) either covalently or noncovalently to an accessory moiety. The accessory moiety can be a therapeutic agent (e.g., exhibiting a biological activity (a “BAM”) or a marker. The BAM can be, for example, a cytotoxin, a non-cytotoxic drug (e.g., an immunosuppressant), a radioactive agent, another antibody, or an enzyme. The marker can be, e.g., any label that generates a detectable signal, such as a radiolabel, a fluorescent label, or an enzyme that catalyzes a detectable modification to a substrate. The nanobody serves a targeting function: by binding to a target tissue or cell where mesothelin is expressed.

In view of the large number of methods that are known for attaching a variety of accessory moieties to antibodies, antibody fragments and antibody-like molecules (including a nanobody or conjugate-construct as disclosed herein) one skilled in the art will be able to determine a suitable method for attaching a given moiety to the nanobody or conjugate-construct. The nanobodies disclosed herein can be derivatized to enable the conjugation. In general, the nanobody or portion thereof is derivatized such that the binding to the target antigen (i.e., mesothelin) is not adversely affected by the derivitization and/or subsequent conjugation.

The nanobodies and conjugate-constructs as disclosed herein can be labeled with a detectable moiety. Useful detection agents include fluorescent compounds, including fluorescein, fluorescein isothiocyanate, rhodamine, 5-dimethylamine-1 -napthalenesulfonyl chloride, phycoerythrin, lanthanide phosphors and the like. Bioluminescent markers are also of use, such as luciferase, Green fluorescent protein (GFP), Yellow fluorescent protein (YFP). A nanobody or conjugate-construct disclosed herein can also be labeled with enzymes that are useful for detection, such as horseradish peroxidase, β-galactosidase, luciferase, alkaline phosphatase, glucose oxidase and the like. When a nanobody or conjugate-construct as disclosed herein is labeled with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. A nanobody or conjugate-construct may also be labeled with biotin, and detected through indirect measurement of avidin or streptavidin binding. It should be noted that the avidin itself can be labeled with an enzyme or a fluorescent label.

A nanobody or conjugate-construct disclosed herein may be labeled with a magnetic agent (such as gadolinium), with lanthanides (such as europium and dysprosium), or with manganese. Paramagnetic particles such as superparamagnetic iron oxide are also of use as labels.

In some embodiments, labels are attached by spacer arms of various lengths to reduce potential steric hindrance.

The nanobodies and conjugate-constructs disclosed herein can also be labeled with a radiolabeled amino acid. The radiolabel may be used for both diagnostic and therapeutic purposes. For instance, the radiolabel may be used to detect the bound mesothelin by x-ray, emission spectra, or other diagnostic techniques. Examples of radioisotopes or radionucleotides include, but are not limited to, ³H, ¹⁴C, ¹⁵N, ³⁵S, ⁹⁰Y, ⁹⁹mTc, ¹¹¹In, ¹²⁵I, and ¹³¹I.

Accessory moieties also include derivitization with a chemical group such as polyethylene glycol (PEG), a methyl or ethyl group, or a carbohydrate group. These groups may be useful to improve the biological characteristics of the nanobody or conjugate-construct, such as to increase serum half-life or to increase tissue binding.

Toxins can be employed as the accessory moiety in the conjugate-constructs disclosed herein. Exemplary toxins include ricin, abrin, diphtheria toxin and subunits thereof, as well as botulinum toxins A through F.

Where a linker is present in the conjugate-construct, e.g., linking the nanobody and accessory moiety such that they are not directly bound to each other, the linker may be cleavable, and may be characterized by their ability to be cleaved at a site in or near a target cell such as at the site of desired therapeutic action or marker activity. Preferred cleavable groups, e.g., by enzymatic cleavage, include peptide bonds, ester linkages, and disulfide linkages. Cleavable linkers may also be sensitive to pH and may be cleaved through changes in pH. In some embodiments, the linker is a peptidyl linker.

### Characterization of Binding to Mesothelin

The molecules and compounds disclosed herein can be tested for binding to mesothelin by any method known in the art or described herein, e.g., standard ELISA. Briefly, microtiter plates or beads are coated with purified and/or recombinant mesothelin protein (see, e.g., Example 1) in PBS, and then blocked with serum albumin in PBS. Dilutions of the molecule to be tested, e.g., a nanobody or conjugate-construct disclosed herein, are contacted with the plate or bead at 37° C. The plates/beads are washed with PBS/Tween and then may be incubated with secondary reagent for detection if necessary.

Reactivity with a mesothelin can also be detected by Western blotting. Briefly, mesothelin or a mesothelin antigen is prepared and subjected to sodium dodecyl sulfate polyacrylamide gel electrophoresis. The separated antigens are transferred to nitrocellulose membranes, blocked with serum, and probed with the (monoclonal) nanobody or conjugate-construct to be tested.

The binding specificity of a nanobody or conjugate-construct disclosed herein can also be determined by monitoring binding of the nanobody or conjugate-construct to cells expressing a mesothelin protein, for example by flow cytometry. Cells or cell lines that naturally express mesothelin protein, such OVCAR3, NC1-H226, CFPAC-1 or KB cells, can be used, or a cell line such as a CHO cell line can be transfected with an expression vector encoding mesothelin such that mesothelin is expressed on the cell surface. The transfected protein may also comprise a tag, such as a myc-tag or a his-tag, preferably at the N-terminus, for detection using an antibody to the tag. Binding of a nanobody or conjugate-construct disclosed herein to a mesothelin protein can be determined by incubating the transfected cells with the nanobody or conjugate-construct, and detecting bound nanobody or conjugate-construct. Binding of an antibody to the tag on the transfected mesothelin may can used as a positive control.

Binding affinity of the nanobodies or conjugate-constructs disclosed herein may be determined according to a BlAcore assay as known in the art or described herein.

### Diagnostic and Therapeutic Methods

The nanobodies and conjugate constructs, and compositions comprising them, have numerous in vitro and in vivo diagnostic and therapeutic utilities involving the diagnosis and treatment of mesothelin-mediated disorders. For example, these molecules can be administered to cells in culture, in vitro or ex vivo, or to human subjects, to treat, prevent, ameliorate, and to diagnose a variety of mesothelin-associated disorders. Preferred subjects include human patients having disorders mediated by mesothelin activity, particularly human patients having a disorder associated with aberrant mesothelin expression. When nanobodies and/or conjugate-constructs to mesothelin are administered together with another agent, the two can be administered in either order or simultaneously.

Given the specific binding of the nanobodies and conjugate constructs disclosed herein for mesothelin, they can be used to specifically detect mesothelin expression. In one embodiment, the compositions molecules and composition of the invention can be used to detect levels of mesothelin, which levels can then be linked to certain disease symptoms. Alternatively, the molecules and compositions can be used to inhibit or block mesothelin function which, in turn, can be linked to the prevention or amelioration of certain disease symptoms, thereby implicating mesothelin as a mediator of the disease. This can be achieved by contacting a sample and a control sample with a nanobody or conjugate construct ad disclosed herein, or a composition comprising such molecules, under conditions that allow for the formation of a complex between the molecules or compositions and mesothelin. Any complexes formed between the molecules or compositions and mesothelin are detected and compared in the sample and the control.

As further detailed herein, the molecules and compositions of the invention have additional utility in therapy and diagnosis of mesothelin-related diseases. For example, the immunoconjugates can be used to elicit in vivo or in vitro one or more of the following biological activities: to inhibit the growth of and/or kill a cell expressing mesothelin; or to block mesothelin ligand binding to mesothelin.

In a particular embodiment, the nanobodies and conjugate-constructs specific for mesothelin disclosed herein, and compositions comprising these molecules, are used in vivo to treat, prevent or diagnose a variety of mesothelin-related diseases. For example, these molecules and compositions can be administered to slow or inhibit the growth of tumor cells or inhibit the metastasis of tumor cells characterized by altered expression of mesothelin. In a preferred embodiment, the nanobody or conjugate-construct specific for mesothelin as disclosed herein is conjugated to a therapeutic agent, such as a cytotoxin. In particularly preferred embodiments, the mesothelin-expressing tumor cell is a mesothelioma cell, or a tumor cell associated with ovarian, pancreatic, stomach, lung, uterine, endometrial, bile duct, gastric/esophageal, colorectal, and breast cancers. In other preferred embodiments, the mesothelin-expressing tumor cell is a mesothelioma cell, a pancreatic tumor cell, an ovarian tumor cell, a stomach tumor cell, a lung tumor cell or an endometrial tumor cell. In still other embodiments, the tumor cell is from a cancer selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas. In these applications, a therapeutically effective amount of a nanobody or conjugate-construct disclosed herein is administered to a subject in an amount sufficient to inhibit growth, replication or metastasis of cancer cells, or to inhibit a sign or a symptom of the cancer. Suitable subjects may include those diagnosed with a mesothelin-associated cancer as disclosed herein.

In one non-limiting embodiment, provided herein is a method of treating a subject with cancer by selecting a subject with a cancer that expresses mesothelin and administering to the subject a therapeutically effective amount of a nanobody or conjugate-construct specific for mesothelin as disclosed herein. Also provided herein is a method of inhibiting tumor growth or metastasis by selecting a subject with a cancer that expresses mesothelin and administering to the subject a therapeutically effective amount of nanobody or conjugate-construct specific for mesothelin as disclosed herein. A therapeutically effective amount of a nanobody or conjugate-construct specific for mesothelin will depend upon the severity of the disease and the general state of the patient's health. A therapeutically effective amount is that which provides either subjective relief of a symptom(s) or an objectively identifiable improvement as noted by the clinician or other qualified observer.

Administration of the nanobody or conjugate-construct specific for mesothelin as disclosed herein can also be accompanied by administration of other anti-cancer agents or therapeutic treatments (such as surgical resection of a tumor). In certain embodiments, the anti-cancer agent is conjugated or linked to the nanobody to form a conjugate construct as described herein. Any suitable anti-cancer agent known in the art can be used in accordance with the invention. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Non-limiting examples of such anti-cancer agents that may be used according to the methods of the invention include, but are not limited to, aldesleukin, alemtuzumab, alitretinoin, allopurinol, altretamine, amifostine, anastrozole, abiraterone, arsenic, axitinib, azacitidine, bendamustine, bexarotene, bleomycin, bortezomib, busulfan, cabazitaxel, calusterone, capecitabine, carboplatin, carmustine, carmustine, celecoxib, chlorambucil, cisplatin, cladribine, clofarabine, crizotinib, cyclophosphamide, cytarabine, dacarbazine, dactinomycin, actinomycin D, dasatinib, daunorubicin, decitabine, dexrazoxane, docetaxel, doxorubicin, epirubicin, eribulin, erlotinib, estramustine, etoposide, everolimus, exemestane, floxuridine, fludarabine, fluorouracil, 5-FU, fulvestrant, gefitinib, gemcitabine, hydroxyurea, idarubicin, lenalidomide, ifosfamide, imatinib, iomustine, irinotecan, isotretinoin, ixabepilone, lapatinib, letrozole, leucovorin, levamisole, lomustine, CCNU, meclorethamine, nitrogen mustard, melphalan, L-PAM, mercaptopurine, 6-MP, mertansine, mesna, methotrexate, methoxsalen, mitomycin, mitotane, mitoxantrone, nandrolone, nelarabine, nilotinib, oxaliplatin, paclitaxel, pamidronate, pazopanib, pegademase, pemetrexed, pentostatin, pipobroman, plerixafor, plicamycin, mithramycin, porfimer, pralatrexate, procarbazine, quinacrine, rapamycin, romidepsin, ruxolitinib, sorafenib, streptozocin, sunitinib, tamoxifen, temozolomide, temsirolimus, teniposide, VM-26, testolactone, thalidomide, thioguanine, 6-TG, thiotepa, topotecan, toremifene, tretinoin, ATRA, uracil mustard, valrubicin, vandetanib, vemurafenib, verteporfin, vinblastine, vincristine, vinorelbine, vismodegib, vorinostat, zoledronate, nucleoside analogues AZT, b-D-arabinofuranose, vidarabine, 2-chlorodeoxyadenosine, intercalating drugs, kinase inhibitors, cofarabine, laromustine, clophosphamide, asparaginase, dexamethasone, prednisone and lestaurtinib. Other anti-cancer treatments include radiation therapy and antibodies that specifically target cancer cells.

The methods of the invention may also be combined with other common anti-cancer treatments, such as, surgical treatment, e.g., surgical resection of the cancer or a portion of it. Another example of a treatment is radiotherapy, for example administration of radioactive material or energy (such as external beam therapy) to the tumor site to help eradicate the tumor or shrink it prior to surgical resection. Anti-cancer treatment according to the invention may be effectively combined with chemotherapeutic regimes. In these instances, it may be possible to reduce the dose of chemotherapeutic reagent administered. Other common combination therapies that may result in synergy with treatment with nanobody or conjugate-construct specific for mesothelin as disclosed herein include hormone deprivation. Angiogenesis inhibitors may also be combined with the treatments disclosed herein.

Methods are also provided herein for detecting expression of mesothelin in vitro or in vivo. In some cases, mesothelin expression is detected in a biological sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

In one embodiment, provided is a method of determining if a subject has cancer by contacting a sample from the subject with a nanobody or conjugate-construct specific for mesothelin as disclosed herein; and detecting binding of the nanobody or conjugate-construct to the sample. An increase in binding of the nanobody or conjugate-construct specific to the sample as compared to binding of the nanobody or conjugate-construct to a control sample identifies the subject as having cancer.

In another embodiment, provided is a method of confirming a diagnosis of cancer in a subject by contacting a sample from a subject diagnosed with cancer with a nanobody or conjugate-construct specific for mesothelin as disclosed herein; and detecting binding of the nanobody or conjugate-construct to the sample. An increase in binding of the nanobody or conjugate-construct to the sample as compared to binding of the nanobody or conjugate-construct to a control sample confirms the diagnosis of cancer in the subject. In certain embodiments, the cancer is mesothelioma cell, or a tumor cell associated with ovarian, pancreatic, stomach, lung, uterine, endometrial, bile duct, gastric/esophageal, colorectal, and breast cancers. In other preferred embodiments, the mesothelin-expressing tumor cell is a mesothelioma cell, a pancreatic tumor cell, an ovarian tumor cell, a stomach tumor cell, a lung tumor cell or an endometrial tumor cell. In still other embodiments, the tumor cell is from a cancer selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas, or any other type of cancer that expresses mesothelin.

In some examples, the control sample is a sample from a subject without cancer. In particular examples, the sample is a blood or tissue sample.

In some cases, the nanobody or conjugate-construct specific for mesothelin is directly labeled with a detectable label. In another embodiment, the nanobody or conjugate-construct specific for mesothelin (first detector) is unlabeled and a second antibody or other molecule that can bind the first detector (the second detector) is labeled.

Suitable labels for a nanobody or conjugate-construct specific for mesothelin as disclosed herein and/or the second detector as described above include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

Mesothelin can be assayed in a biological sample by a competition immunoassay utilizing mesothelin standards labeled with a detectable substance and an unlabeled nanobody or conjugate-construct specific for mesothelin as disclosed herein. In this assay, the biological sample, the labeled mesothelin standards and the nanobody or conjugate-construct specific for mesothelin are combined and the amount of labeled mesothelin standard bound to the unlabeled nanobody or conjugate-construct specific for mesothelin is determined. The amount of mesothelin in the biological sample is inversely proportional to the amount of labeled mesothelin standard bound to the nanobody or conjugate-construct specific for mesothelin.

The assays and methods disclosed herein can be used for a number of purposes. In one embodiment, the nanobody or conjugate-construct specific for mesothelin as disclosed herein may be used to detect the production of mesothelin in cells in cell culture. In another embodiment, the nanobody or conjugate-construct specific for mesothelin as disclosed herein can be used to detect the amount of mesothelin in a biological sample, such as a tissue sample, or a blood or serum sample. In some examples, the mesothelin is cell-surface mesothelin; in other examples, the mesothelin is soluble mesothelin (e.g., mesothelin in a cell culture supernatant or soluble mesothelin in a body fluid sample, such as a blood or serum sample).

In one embodiment, a kit is provided for detecting mesothelin in a biological sample, such as a blood sample or tissue sample, e.g., to confirm a cancer diagnosis in a subject. A biopsy can be performed to obtain a tissue sample for histological examination according to this method. Alternatively, a blood sample can be obtained to detect the presence of soluble mesothelin protein or fragment. Kits for detecting a polypeptide will typically comprise a (monoclonal) nanobody or conjugate-construct specific for mesothelin such as any such molecule disclosed herein.

In one embodiment, a kit includes instructional materials disclosing means of use of a nanobody or conjugate-construct specific for mesothelin as disclosed herein. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting mesothelin in a biological sample generally includes the steps of contacting the biological sample with a nanobody or conjugate-construct as disclosed herein that specifically reacts with mesothelin under immunologically reactive conditions. The nanobody or conjugate-construct specific for mesothelin is allowed to specifically bind under immunologically reactive conditions to form an immune complex, and the presence of the immune complex is detected directly or indirectly.

As has been detailed herein, the invention provides nanobodies and conjugate constructs specific for mesothelin as well as the diagnostic and therapeutic use thereof, e.g., in the diagnosis, prevention, treatment and/or amelioration of cancer or a symptom thereof Exemplary anti-mesothelin nanobodies include nanobodies having the amino acid sequence SEQ ID NO:1 or SEQ ID NO:2 (Nb-A1 or Nb-C6, respectively). The nanobodies as disclosed herein can be isolated from a phage library derived from B cells of immunized llamas. Despite monovalent binding, the nanobodies (and conjugate constructs based thereon) have high affinity, e.g, a K_{D} less than 5×10⁻⁸ M with exemplary nanobodies Nb-A1 and Nb-C6 having an apparent K_{D} of approximately 15 nM and 30 nM, respectively. The higher affinity and maximum MFI achieved with Nb-A1 is consistent with its predominant representation in the phage display output and the larger fluorescence shift seen with flow cytometry as shown in the Examples, below. The combined flow cytometry, immunofluorescence, western blot, and nanoparticle targeting results with the exemplary nanobodies show that the nanobodies and conjugate-constructs as provided herein can provide a flexible approach to phenotype tumors using conventional diagnostic techniques prior to incorporating the nanobody or conjugate-construct into novel immunotargeting-based diagnostic and therapeutic nanotechnologies.

In addition to the diagnostic applications described and exemplified herein, conjugated nanobodies disclosed herein can be conjugated (e.g., to form conjugate-constructs) into nanosensors that recognize mesothelin (e.g., the biotinylated or cysteine-containing nanobodies such a that comprising SEQ ID NO:1 (Nb-A1)). The sensitivity of immunosensors depends critically on the amount and functionality of the immobilized antibody or antibody fragment (e.g., the nanobody or conjugate-construct as disclosed herein). Since site-specific immobilization produces nanoparticles or surfaces with a higher density of antigen binding sites in a productive orientation for antigen recognition (see, e.g., Sukhanova et al., Nanomedicine 8(2012), 516-525; Loch et al., Mol Oncol 1(2007), 313-320), the higher density of functional antibody fragments possible via site-directed coupling of the nanobodies and conjugate-constructs disclosed herein compared with natural IgG enhances the nanosensor response and decreases the detection limit. For example, an immunosensor prepared according to the methods disclosed herein and using the nanobodies and conjugate-constructs of the invention was able to recognize osteopontin, a prostate cancer biomarker, at a concentration of 1 pg/mL or 30 fM which is three orders of magnitude more sensitive than an ELISA, see, Lerner et al., ACS Nano 6(2012), 5143-5149.

The availability of high affinity anti mesothelin nanobodies and conjugate-construct compatible with a variety of oriented coupling approaches represents an important step toward the generation of mesothelin specific immunosensors with comparable sensitivity, which would have direct and immediate implications in the early detection and prognosis of ovarian cancer. Finally, the availability of a small mesothelin targeting domain opens the possibility to generate next generation therapeutic molecules such as bispecific nanobodies or immunotoxins; see, Rozan et al., Mol Cancer Ther 12(2013), 1481-1491; Weldon et al., Mol Cancer Ther 12(2013), 48-57.

The molecules and methods of the present invention may be used to detect native and denatured mesothelin in various diagnostic applications, including flow cytometry, western blotting, immunofluorescence, and optical imaging. The anti-mesothelin nanobodies and conjugate constructs disclosed herein are novel, cost-effective, small, and single domain reagents with high affinity and specificity for the tumor-associated antigen mesothelin, which can additionally be easily bioengineered for attachment to nanoparticles or modified surfaces using multiple bioconjugation strategies. The anti-mesothelin nanobodies and conjugate constructs disclosed herein are useful in both conventional and nanotechnology-based diagnostic, therapeutic and prognostic biomedical applications.

So that the manner in which the above-recited features, aspects and advantages of the invention, as well as others that will become apparent, are attained and can be understood in detail, more particular description of the invention briefly summarized above can be had by reference to the embodiments thereof that are illustrated in the drawings that form a part of this specification. It is to be noted, however, that the appended drawings illustrate some embodiments of the invention and are, therefore, not to be considered limiting of the invention's scope, for the invention can admit to other equally effective embodiments.

The present disclosure and invention is further illustrated by the following examples, which should not be construed as further limiting. The contents of all documents, references, Gen-bank sequences, patents and published applications cited throughout this application are hereby expressly incorporated by reference herein in their entirety.

### EXAMPLES

### Materials and Methods

### Llama Immunization and VHH Library Construction

A young adult male llama (Lama glama) was immunized subcutaneously at days 1, 20, 41 and 62 with 65 μg of recombinant human, soluble mesothelin protein produced as previously described; Scholler et al., Cancer Lett 247(2007), 130-136. The VHH library was constructed as previously described; Behar et al., Febs J276(2009), 3881-3893.

### Selection of Nanobodies by Phage Display

Phages from the VHH library were produced as previously described; Behar et al, Protein Eng Des Sel 21(2008), 1-10. Mesothelin conjugated to epoxy-coated paramagnetic beads (Dynabeads M-450 Epoxy, Invitrogen) were used for two sequential rounds of immunoselection to identify phages that specifically recognize mesothelin. To label the Dynabeads, an aliquot (100 μL) was washed with 0.1 M sodium phosphate buffer (NaPi) and resuspended in 100 μL of NaPi. Recombinant mesothelin (10 μg; Bergan et al., Cancer Lett 255(2007), 263-274) was added to the beads and the solution was gently rotated for 48 h at 4° C. Beads were washed three times by magnetic isolation with 1 mL of PBS/0.1% Tween-20 and then three times with 1 mL of PBS before being incubated with 1 mL of PBS/2% milk for 2 h at room temperature. Mesothelin conjugated beads were resuspended with the phage preparation pre-incubated in PBS/2% milk. The solution was gently rotated for 2 h at room temperature before being washed nine times with PBS/0.1% Tween-20, nine times with 1 mL of PBS, and then incubated with 500 μL of trypsin (1 mg/mL) for 30 min at room temperature. Eluted phage-nanobodies were resuspended in 500 μL of PBS and incubated without shaking with 5 mL of log phase TG1 cells which were subsequently plated on 2YT/ampicillin (100 μg/mL)/2% glucose (2YTAG) in 243×243 dishes (Nalgene Nunc). Ninety three colonies from the first round of selection and 192 colonies from the second round of selection were picked, grown overnight in 96-well plates containing 200 μL 2YTAG and stored at −80° C. after the addition of 15% glycerol. The remaining colonies were harvested from the plates, suspended in 5 mL of 2YTAG and used to produce phages for the next round of selection.

### ELISA Screening of Phage-Nanobodies

Infected TG1 cells (5 μL) from masterplates were used to inoculate 150 μL of 2YTAG in 96-well plates. Colonies were grown for 2 h at 37° C. under shaking (900 rpm) then 50 μL of 2YT containing 2×10⁸ M13K07 helper phage were added to each well and incubated for 30 min at 37° C. without shaking. Plates were centrifuged for 10 min at 1200×g and bacterial pellets were resuspended in 150 μL of 2YT containing ampicillin (100 μg/mL) and kanamycin (50 μg/mL), 2YTAK. Colonies were grown for 16 h at 30° C. under shaking (900 rpm). Phage-containing supernatants were tested for binding to recombinant mesothelin by ELISA. Fifty micrograms of mesothelin were biotinylated in vitro using the EZ-Link Micro NHS-PEO4-Biotinylation Kit (Pierce) according to the manufacturer's recommendations. Biotinylated recombinant mesothelin (1.4 μg/mL) was bound to streptavidin-coated 96-well microplates for 16 h with PBS/2% milk. Fifty microliters of phage supernatant was added to 50 μL PBS/2% milk and incubated for 1 h at room temperature in the ELISA microplate. Bound phages were detected at A₄₀₅ using a peroxidase-conjugated monoclonal anti-M13 mouse IgG.

### Cell Culture

The human cervix adenocarcinoma HeLa cell line was obtained from the American Type Culture Collection (ATCC) and was cultured in Dulbecco's modified Eagle's medium supplemented with 10% heat-inactivated fetal bovine serum (FBS). Jurkat cells from ATCC were cultured in RPMI-1640 with 10% FBS. The SK-OV-3 and OVCAR-3 human ovarian adenocarcinoma cell lines were obtained from ATCC and cultured in DMEM with 10% FBS and RPMI-1640 with 20% FBS, respectively. The 22Rv1 human prostate carcinoma cell line was a kind gift of Raphael Scharfmann and was cultured in RPMI-1640 with 10% FBS. Ovarian cancer cell lines (C30 and A1847) from the University of Pennsylvania Ovarian Cancer Research Center were cultured in RPMI-1640 media with 10% FBS containing 1% penicillin/streptomycin (100 Units/mL penicillin and 100 μg/mL streptomycin). Human embryonic kidney 293 cells from ATCC, which were transfected to secrete a chimeric protein containing the extracellular portion of mesothelin and an IgG hinge (293-Msln-Ig), were cultured in DMEM media with 10% FBS containing 50 μg/mL hygromycin B and 1% penicillin/streptomycin as described previously; Bergan et al., Cancer Lett 255(2007), 263-274. All cell lines were maintained at 37° C. under a humidified 5% CO₂ atmosphere.

### Screening of Phage-Nanobodies on Mesothelin-Positive Cells by Flow Cytometry

Phage-containing supernatants were tested for binding to HeLa cells (mesothelin positive) and Jurkat cells (mesothelin negative). Flow cytometry was performed after incubating 5×10′ cells with 50 μL of phage-containing supernatants for 1 h at 4° C. under shaking (900 rpm). Phage binding was detected by incubation with a primary monoclonal anti-M13 mouse IgG (10 μg/mL, GE Healthcare Life Sciences) followed by a phycoerythrin (PE)-labeled F(ab)′₂ goat anti-mouse IgG (H+L) secondary antibody (Santa Cruz Biotechnology). Analyses were carried out using a MACSQuant® Analyzer (Miltenyi Biotec) with FlowJo software. Phages displaying mean fluorescence intensity (MFI) two times above the negative control were considered as mesothelin-specific phages.

### Nanobody Sequencing, Production and Purification

DNA sequences of mesothelin-specific phages were determined by GATC Biotech AG (Applied Biosystems). One nanobody from each identified family was selected, produced in E. coli strain BL21DE3, and subsequently purified. Overnight cultures in 2YTAG were diluted into 2YT (50 mL) supplemented with 2 mM MgSO₄, 0.05% glucose, 0.5% glycerol, 0.2% lactose and 100 μg/mL ampicillin to obtain an OD600 of 0.1. Bacteria were grown for 2 h at 37° C. then for 16 h at 30° C. under shaking (900 rpm). Cells were harvested by centrifugation at 3000×g for 20 min at 4° C. and the pellet was kept overnight at −20° C. The pellet was resuspended in 5 mL of room temperature Bug Buster Extraction Reagent (Novagen) supplemented with 10 μL of lysozyme (10 mg/mL) and 0.5 μL of benzonase (250 U/μL). After incubation for 30 mM at room temperature with gentle shaking, nanobodies were purified by TALON metal-affinity chromatography (Clontech) and concentrated by ultrafiltration with Amicon Ultra 5000 MWCO (Millipore). The protein concentration was determined spectrophotometrically using the Bio-Rad DC protein assay (Bio-Rad Laboratories).

### Cell Binding Experiments by Flow Cytometry

Nanobodies and the anti-mesothelin mouse monoclonal antibody K1 (mAb K1, Santa Cruz Biotechnology) were used to perform cell binding experiments by flow cytometry. Immunofluorescence assays were performed by incubating 5×10⁵ indicator cells (SK-OV-3, OVCAR-3, or 22Rv1) with Nb A1 (0.5 82 g/mL; comprising the amino acid sequence SEQ ID NO:1), Nb C6 (0.5 μg/mL; comprising the amino acid sequence SEQ ID NO:2) or mAb K1 (0.4 μg/mL) for 1 h at 4° C. with shaking (900 rpm). Nanobody binding to each cell line was detected by incubation with a mouse F(ab)′₂ anti-6His antibody (1 μg/mL) followed by phycoerythrin-goat anti mouse IgG antibody (PE-GAM). An irrelevant nanobody was used as a negative control. Binding of mAb K1 was detected by incubation with PE-GAM. PE-GAM was directly used as a negative control.

### Immunofluorescence Competition Assay

Competition assays between nanobodies comprising SEQ ID NO:1 and SEQ ID NO:2 (i.e., Nb-A1 and Nb-C6, respectively) were performed by incubating 5×10⁵ HeLa cells with various concentrations of Nb-A1 (from 0.5 pM to 5 μM) and a 1/200 dilution of the phage-Nb-C6. The same experiment was performed with various concentrations of Nb-C6 (from 0.5 pM to 5 μM) and a 1/500 dilution of the phage-Nb-A1. The binding of phage-Nbs was detected by incubation with monoclonal anti-M13 mouse IgG (10 μg/mL) followed by incubation with PE-GAM. The same experiment was performed with a 1/100 dilution of commercial K1 antibody as positive control. The binding of mAb K1 was detected by incubation with PE-GAM.

### Affinity Measurements of Nb

Briefly, 50 μg of each nanobody and mAb K1 were chemically biotinylated using the EZ-Link Micro NHS-PEO4-Biotinylation Kit. After incubation of mesothelin-positive HeLa cells (5×10⁵) with various concentrations of biotinylated antibodies for 1 h at 4° C. under shaking (900 rpm), antibody binding was detected by flow cytometry following incubation with (PE)-labeled streptavidin. The K_{D} values were determined by the equation: 1/(F−F_{back})=1/Fₘₐₓ+(K_{D}/Fₘₐₓ)(₁/[antibody]), in which F represents the fluorescence unit, F_{back}=background fluorescence and Fₘₐₓ is estimated from the data. The slope of the regression line is (a)=K_{D}/Fₘₐₓ so K_{D}=a*Fₘₐₓ; see, Even-Desrumeaux et al., Methods Mol Biol 907(2012), 443-449.

### Cloning and Expression of Soluble, Site-Specific Biotinylated Nanobody, Nb-A1

Site-specifically biotinylated nanobody A1 (named Bb A1) was derived from the nanobody comprising the sequence SEQ ID NO:1 (Nb-A1) and was biosynthetically produced following an established protocol developed for scFv; see, e.g., Scholler et al., J Immunol Methods 317(2006), 132-143; Zhao et al., J Immunol Methods 363(2011), 221-232. Briefly, the Nb-Al sequence (amino acid sequence SEQ ID NO:1, e.g., encoded by SEQ ID NO:3) was PCR amplified to incorporate terminal sequences for homologous recombination with the p416-BCCP vector containing a biotin ligase recognition sequence. Linearized p416-BCCP vector and PCR product were chemically transformed into haploid Saccharomyces cerevisiae cells (YVH10) which were subsequently mated with haploid yeast containing a plasmid coding for the Escherichia coli biotin ligase for antibody secretion into the yeast culture supernatant after galactose induction. The site-specifically biotinylated molecules are named biobodies (Bb).

### Sodium Dodecyl Sulfate Polyacrylamide Gel Electrophoresis (SDS-PAGE) and Western Blotting of 293-Msln-Ig Culture Supernatant

To obtain a chimeric protein containing the extracellular portion of mesothelin fused to an IgG hinge, 293-Msln-Ig cells were grown to confluency, washed with DPBS, incubated in DMEM lacking FBS until the cells started to detach, and the culture supernatant was clarified by centrifugation. Culture supernatant (2 pig) in reducing sample buffer was loaded on a SDS-PAGE gel, along with high range rainbow molecular weight markers (GE Healthcare). Proteins were transferred from the SDS-PAGE gel to an Immobilon-P PVDF transfer membrane (Millipore) using a Mini Trans-Blot module (Bio-Rad) for 1 h at 70 V. The membrane was blocked overnight with Superblock T20 PBS blocking buffer (Thermo Scientific). To detect mesothelin, blots were incubated with either Bb A1 or K1 (Santa Cruz Biotechnology) at 2 μg/mL in Superblock for 1 h at room temperature. The blots were washed three times with PBST (PBS containing 0.05% (v/v) Tween-20) and were incubated for 30 min with a 1:20,000 dilution of streptavidin-HRP (BD Pharmingen) in Superblock to detect Bb A1 or a 1:10,000 dilution of anti-mouse IgG HRP (GE Healthcare) in Superblock for 1 h to detect K1. The Ig hinge on Msln-Ig was directly detected with a 1:10,000 dilution of HRP conjugated F(ab′)2 goat anti-human IgG (H+L) from Jackson Immunoresearch using a similar protocol. The blots were washed three times with PBST and detected with Luminata Classico Western HRP substrate (Millipore) using double emulsion blue basic autoradiography film (GeneMate).

### Self-Assembly of Targeted Superparamagnetic Iron Oxide Nanoparticles (SPION) for Flow Cytometry

The self-assembly of immunotargeted, fluorescent nanoparticles was performed according to a previously published protocol; Prantner et al., In Targeting of superparamagnetic iron oxide nanoparticles for cancer therapy based on localized hyperthermia, 6th annual Symposium Center for Translational Medicine, Jefferson Medical College, Philadelphia, Pa., Jefferson Medical College, Philadelphia, Pa., 2010. Briefly, superparamagnetic iron oxide nanoparticles conjugated to streptavidin (SA-SPION) (5 μL, MagCellect streptavidin ferrofluid, R&D Systems) were added to DPBS containing 5 mg/mL bovine serum albumin (500 μL, DPBS-BSA) in polystyrene round bottom tubes, mixed by vortexing, and magnetically separated using a DynaMag-2 magnet (Invitrogen) for 10 min. The fluid was removed and replaced with YCS containing Bb Al (500 μL), supplemented with 15 ng/mL biotin-4-fluorescein (B4F, Invitrogen) for staining and 10 M sodium hydroxide (2.5 μL into 500 μL YCS) to adjust the pH. The complexes were incubated for 30 min at room temperature in the dark, magnetically separated for 10 min, and washed two times with 500 μL DPBS-BSA. After the final wash, the complexes were resuspended in DPBS containing 1% fetal calf serum for flow cytometry analysis.

### Flow Cytometry Using Fluorescent SA-SPION

Ovarian cancer cell lines of human origins (A1847 and C30) were grown on tissue culture-treated plates and non-enzymatically detached by pipet mixing with a PBS-based, enzyme-free cell dissociation buffer (5 mL, Gibco). Then, 10⁵ cells were incubated with the appropriate nanoparticle preparation (500 μL), a mouse IgG1 isotype control (5 μg/mL), or mAb K1 (5 μg/mL) for 30 min at 4° C., washed twice with DPBS containing 1% FBS (500 μL, PBS-FBS) and resuspended in PBS-FBS (500 μL). Prior to flow cytometry, 7-amino-actinomycin D (Via-Probe, Becton Dickinson) was added to identify viable cells for subsequent analysis of the fluorescein fluorescence intensity.

### Tumor Spheroid Preparation and Immunofluorescence

Tumor spheroids were generated using a liquid overlay technique (see, Carlsson et al, Recent Res Cancer 95(1984), 1-23) modified as follows. Ninety-six-well plates were coated with 1.6% agarose (50 μL) and allowed to solidify. Human ovarian cancer cells (A1847) were detached from a T25 flask with 0.05% trypsin/EDTA (Gibco) and resuspended in RPMI media containing 10% FBS and 1% penicillin/streptomycin at a cell density of 5×10⁵ cells/mL. Cells (200 μL) were applied to agarose-coated wells and maintained at 37° C. under a humidified 5% CO₂ atmosphere while rotating at 120 rpm for 2 days. Tumor spheroids were then washed with PBS (500 μL). For frozen sections, the spheroids were placed in the bottom of a cryomold, optimal cutting temperature (OCT) compound was added, and the samples were frozen on dry ice for sectioning. The sections were dried at room temperature for 30 min, fixed at room temperature for 10 min using acetone pre-cooled to −20° C., and then washed three times for 5 min in wash buffer (Dako). The slides were blocked for 30 min with serum-free protein block (Dako). Bb A1 (10 μg/mL) diluted in antibody diluent (Dako) was incubated on the slides overnight at 4° C. in a humidified chamber. The slides were washed three times for 5 min in wash buffer before adding Alexa Fluor 488-labeled anti-V5 mAb (1:100 dilution, AbD Serotec) for 1 h. Slides were counterstained with DAPI, washed three times for 5 min with wash buffer, and mounted with Fluoromount-G (SouthernBiotech). For fixed, paraffin embedded sections, the spheroids were placed in formalin for 1 h, dehydrated through an ethanol gradient, and embedded in paraffin for sectioning. After mounting, slides were heated to 60° C. for 20 min, cooled to room temperature, washed twice in xylene for 15 min, rehydrated through an ethanol gradient into water. Antigen retrieval was performed using high pH antigen unmasking solution (Vector Labs). Slides were washed two times for 5 min in PBS and then once in wash buffer for 5 min. The slides were blocked for 30 min with serum-free protein block (Dako). Bb A1 (10 μg/mL) in antibody diluent (Dako) was incubated on the slides overnight at 4° C. in a humidified chamber. The slides were washed three times for 5 min in wash buffer before adding Alexa Fluor 488-labeled anti-VS (1:100 dilution, AbD Serotec) for 1 h. Slides were counterstained with DAPI, washed three times for 5 min with wash buffer, and mounted with Fluoromount-G (SouthernBiotech). Negative controls for both the frozen and paraffin sections used the same protocol except that the slides were incubated overnight with antibody diluent instead of Bb A1. Spheroid sections were imaged with a Zeiss Axioplan upright microscope and processed using ImageJ.

### Quantum DotLlabelling with Cys-A1

Cys-A1 was derived from Nb-A1 (comprising amino acid sequence SEQ ID NO:1) with standard molecular biology protocols to include a cysteine for thiol-maleimide coupling. Purified Cys-A1 was coupled to a quantum dot using a Qdot 800 antibody conjugation kit (Invitrogen) according to the manufacturer's instructions. Cells (A1847 and C30) were grown on 8-well chamber slides (Lab-Tek II CC², Nunc) and labeled with carboxyfluorescein diacetate, succinimidyl ester (CFSE, Invitrogen) in PBS for 15 min at 37° C. Then, cells were washed, incubated for an additional 30 min in cell culture media, and fluorescently labeled; see, Willingham et al., Methods Mol Biol 115(1999), 113-119. Briefly, cells were first washed with 500 μl of DPBS containing calcium and magnesium (PBS++) and blocked for non-specific binding for 5 min at 4° C. with PBS++ supplemented with 2 mg/mL bovine serum albumin (Sigma-Aldrich), BSA-PBS++. Qdots labeled with Cys-A1 were diluted to either 10 or 50 nM in BSA-PBS++(200 μL) at 4° C. and were added to the cells and incubated for 30 min at 4° C. in the dark. Unbound Qdots were removed by aspiration and the cells were washed three times with BSA-PBS++ (500 μL) at 4° C. followed by a wash with room temperature PBS++ (500 μL). Cells were mounted with Fluoromount G (Southern Biotech). The slides were imaged on an IVIS Spectrum pre-clinical in vivo imaging system (Perkin Elmer) using excitation/emission wavelengths of 500/540 nm for CFSE and 430/800 nm for Qdot 800.

### Immunofluorescence of Nb-A1 Binding at Physiological Temperature

Cells (C30, A1847, and Hela) were allowed to grow to confluence in a 24-well tissue culture plate. Once the cells were confluent, two drops of OneComp eBeads (eBioscience) were incubated with mouse anti-V5:Alexa Fluor488 (2 μg) for 30 min at room temperature in the dark. The beads were washed twice with 1% BSA in PBS (1 mL) by centrifugation at 600×g for 5 min. The beads were resuspended in 50 μL of 1% BSA in PBS++ and incubated in the dark with 2 μg of Bb A1 for 45 min at room temperature. The beads washed twice with 1% BSA in PBS++ (1 mL) by centrifugation at 600×g for 5 min, resuspended in growth media containing 10% FBS, and incubated at 37° C. for 4 hr. At the end of the incubation, the cells were washed twice with 500 μL of PBS++, fixed with HistoChoice (Sigma) for 15 min at room temperature in the dark, washed three times with 500 μL of PBS++, and the nuclei were stained with 1 μg/mL Hoechst 33258 (Invitrogen). Forty-two fluorescent images per well were collected using an EVOS FL Auto cell imaging system (Invitrogen) at 10× magnification. ImageJ was used to analyze the fluorescent images.

### Statistical Analysis

A two-tailed Student's t-test in Excel was used to calculate the probability that the mean number of particles bound to C30, A1847, and Hela cells were different.

### EXAMPLE 1

### Selection of Anti-Mesothelin Nanobodies by Phage Display

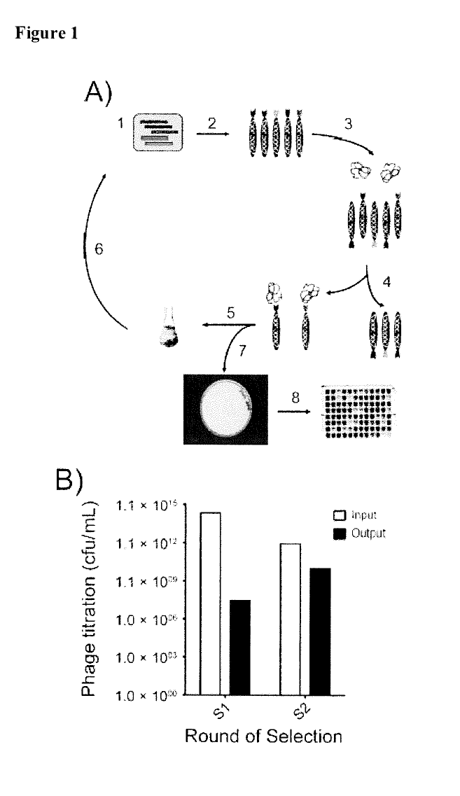

FIG. 1A presents a schematic of the phage-display method by which nanobodies were selected for mesothelin specificity from camelid immunoglobulin libraries. A nanobody library (˜10⁸ clones) was constructed using peripheral blood cells of llama immunized with recombinant mesothelin. Two rounds of direct selection using phage antibody produced with helper phage KM13 was used to pan over epoxy-coated paramagnetic beads previously incubated with mesothelin. Enrichment in the number of phages that recognize mesothelin could be detected between the first and second round of selection (FIG. 1B). Accordingly, a phage-ELISA based screening procedure performed after the first round of affinity selection using biotinylated mesothelin immobilized on streptavidin plates revealed that 82 out of 93 clones (88%) were positive. After the second round of selection, all clones picked from the output recognized mesothelin and produced background signals on control antigens. Forty-five out of 93 clones were assayed by flow cytometry for binding to mesothelin expressed on the plasma membrane of HeLa cells (mesothelin positive) or to Jurkat cells (mesothelin negative). Thirty-seven out of 45 clones bound exclusively to HeLa cells. Sequence analyses of the 20 clones displaying the highest mean fluorescence intensities revealed 2 independent nanobodies: Nb-A1 (comprising amino acid sequence SEQ ID NO:1; representing 95% of binders) and Nb-C6 (comprising amino acid sequence SEQ ID NO:2; representing 5% of binders). Nb-A1 comprised a VHH CDR1 having the sequence SEQ ID NO:7, a VHH CDR2 having the sequence SEQ ID NO:8, and a VHH CDR3 having the sequence SEQ ID NO:9. Nb-C6 comprised a VHH CDR1 having the sequence SEQ ID NO:10, a VHH CDR2 having the sequence SEQ ID NO:11, and a VHH CDR3 having the sequence SEQ ID NO:12. The presence of an arginine on position 45 confirmed the camelidae nature of these single domain antibodies; Harmsen et al., Mol Immunol 37(2000), 579-590.

### EXAMPLE 2

### Binding Specificity of Nanobodies to Mesothelin Positive Cells

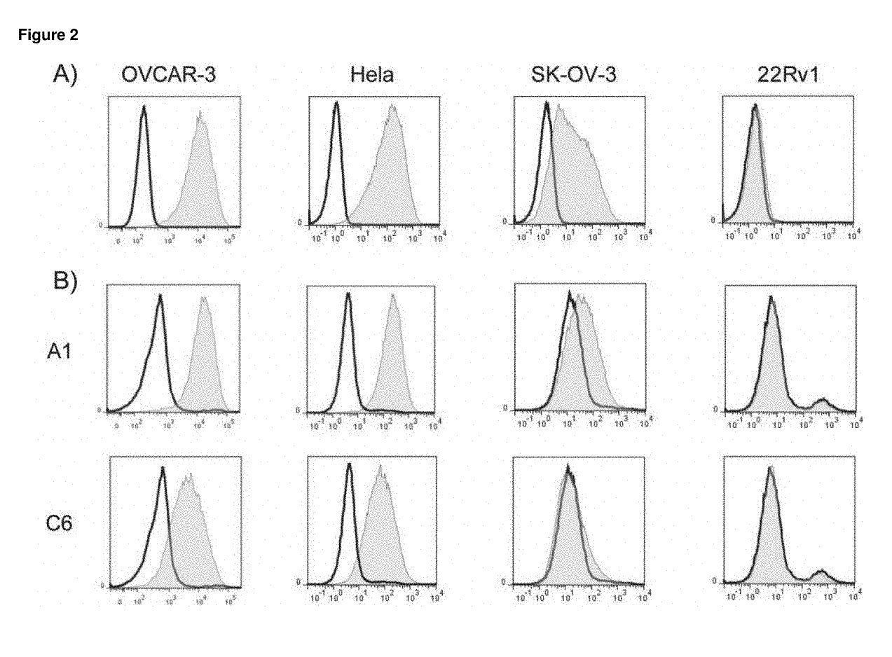

The nanobody specificity was further characterized by flow cytometry on cell lines with different mesothelin expression levels. Nanobodies containing a C-terminal hexahistidine tag were produced in the periplasm of E. coli and purified by immobilized ion metal affinity chromatography. Final yields were in the range of 50 mg/L culture for the two clones, Nb-A1 and Nb-C6. SDS-PAGE analysis demonstrated a satisfying degree of purity (>95%, data not shown). Nanobodies were assayed by flow cytometry for binding to ovarian cancer cells (OVCAR-3 and SK-OV-3), cervix adenocarcinoma cells (HeLa) or to prostate carcinoma cells (22Rv1). Mesothelin expression was initially assessed on each cell line using the commercially available anti-mesothelin monoclonal antibody K1 (FIG. 2A). The mAb K1 binding profiles confirmed that OVCAR-3 and HeLa cells over-express mesothelin. The ovarian cancer SK-OV-3 cell line showed a moderate mesothelin expression while the prostate carcinoma cell line 22Rv1 did not express a detectable antigen level. Importantly, Nb-A1 binding profiles were similar to mAb K1 despite its monovalency (FIG. 2B). Cell binding was also observed with Nb-C6, but to a lesser extent than Nb-A1 since no binding was observed on the SK-OV-3 cell line that expresses moderate levels of mesothelin. No binding of mAb K1, Nb-A1, or Nb-C6 was detected on the prostate carcinoma cell line 22Rv1. Taken together, these results confirmed that both clones specifically bound mesothelin.

### EXAMPLE 3

### Competitive Mesothelin Binding Assay

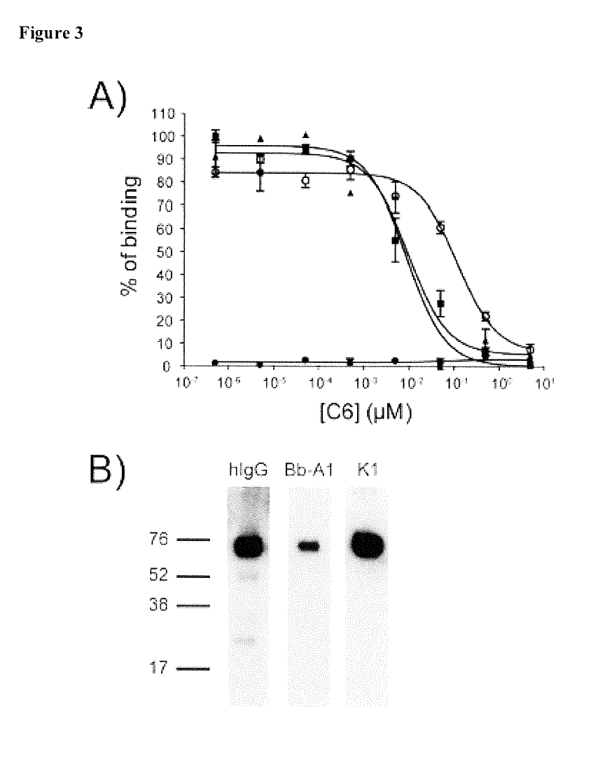

To determine if Nb-A1 and Nb-C6 recognize the same or overlapping epitopes, the phage-nanobodies (phage-Nbs) A1 and C6 and an irrelevant phage-Nb were assayed by flow cytometry for binding to HeLa cells in the presence of serial dilutions of purified Nb-C6. As expected, phage-Nb-C6 competed with Nb-C6 (FIG. 3A). A competitive binding was also observed between phage-Nb-A1 and Nb-C6, which indicates that the two clones bind the same or a proximal mesothelin epitope. The same result was obtained by the reverse experiment, which assayed phage-Nb-C6 binding to HeLa cells in the presence of serial dilutions of Nb-A1 (data not shown), confirming the competition between the two clones. A competition was also observed using mAb K1 and serial dilutions of purified Nb-C6 (FIG. 3A). The same result was observed with Nb-A1 (data not shown) indicating that the epitope bound by Nb-A1 and Nb-C6 is the same one recognized by mAb K1. To further characterize this common epitope, an immunoblot was performed using mammalian cell culture supernatant containing a recombinant human mesothelin (Msln-Ig) fusion protein. After reducing SDS-PAGE and transfer to PVDF membrane, the recombinant Msln-Ig was detected using Nb-Al and mAb K1. Detection of the recombinant protein by anti human IgG (H+L) antibody was used as positive control. As seen in FIG. 3B, the three antibodies detected the same band, which indicates that both mAb K1 and the Nb-A1 recognize a linear epitope. These results also establish that Nb-A1 can be used for immunobloting procedures.

### EXAMPLE 4

### Affinity Determination of Anti-Mesothelin Nanobodies on Cells

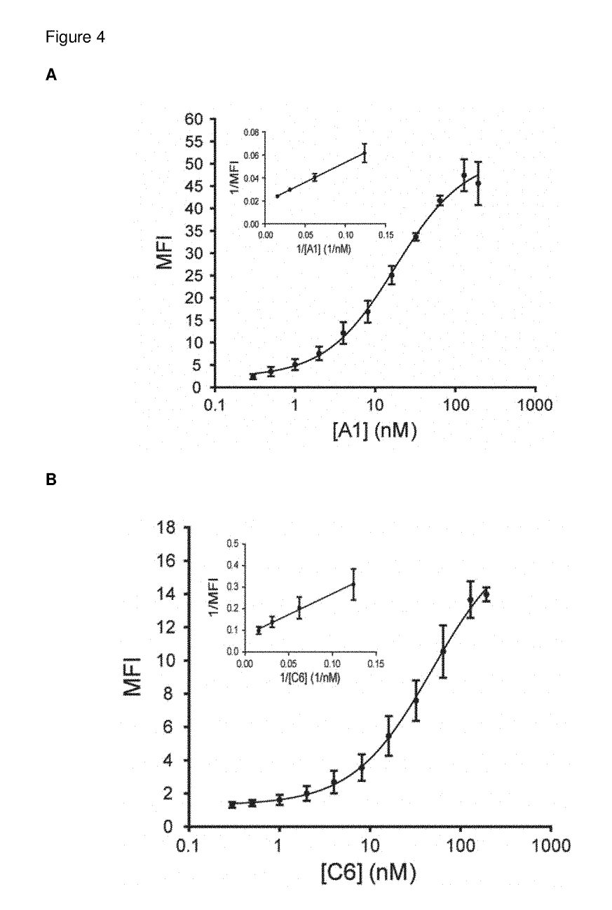

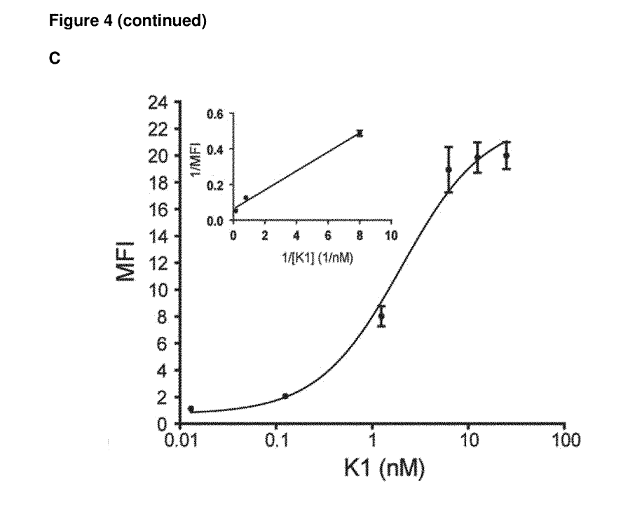

The affinity of Nb-A1 and Nb-C6 for cellularly expressed mesothelin was determined by flow cytometry using HeLa cells for the antigen as described previously; Even-Desrumeaux et al., Mol Biosyst 8(2012), 2385-2394. Briefly, binding to HeLa cells was detected using flow cytometry after incubation with various concentrations of biotinylated nanobodies followed by PE-labeled streptavidin. Apparent K_{D} values were determined by the equation K_{D}=a*Fₘₐₓ in which (a) is the regression line and Fₘₐₓ is the maximum of fluorescence. Despite their monovalency, Nb-A1 had an apparent K_{D} of approximately 15 nM while Nb-C6 had an apparent K_{D} of 30 nM (FIG. 4). For comparison, the bivalent commercial mAb K1 had an apparent K_{D} of approximately 2 nM using the same experimental conditions. Based on these results and those obtained in the competition assay, subsequent experiments were performed using only the representative Nb-A1.

### EXAMPLE 5

### Immunofluorescence Detection of Mesothelin

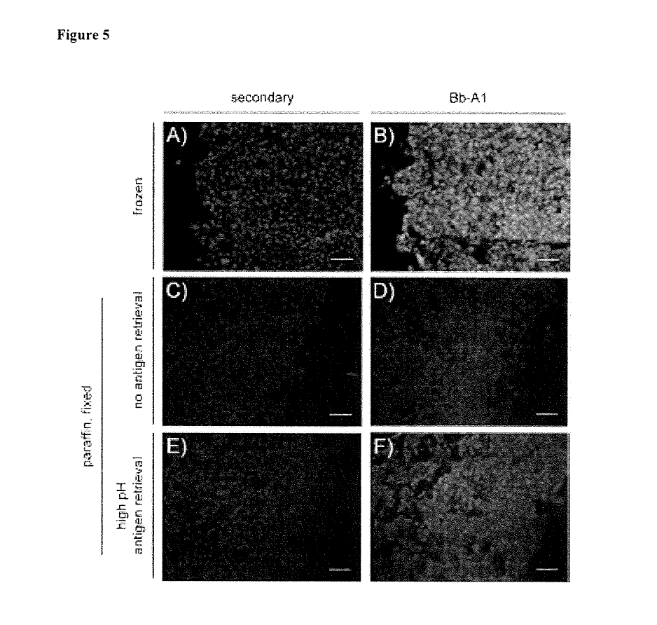

Clinical application of personalized medicine in cancer therapy using novel molecularly targeted platforms requires reliable tumor phenotyping. The reactivity of biobody Bb A1, a metabolically and site-specifically biotinylated version of Nb-A1 was detected using immunofluorescence assays with frozen or formalin fixed, paraffin embedded sections from a multicellular tumor spheroid. Biobody A1 specifically and efficiently recognized mesothelin in frozen sections (FIGS. 5A and B) compared to a control section incubated with only the secondary antibody. In contrast, Bb A1 showed poor reactivity on fixed, paraffin embedded sections prior to antigen retrieval (FIGS. 5C and D), which could be significantly enhanced by antigen retrieval at high pH (FIGS. 5E and F).

### EXAMPLE 6

### Nanobody Mediated Targeting of Nanoparticles to Mesothelin

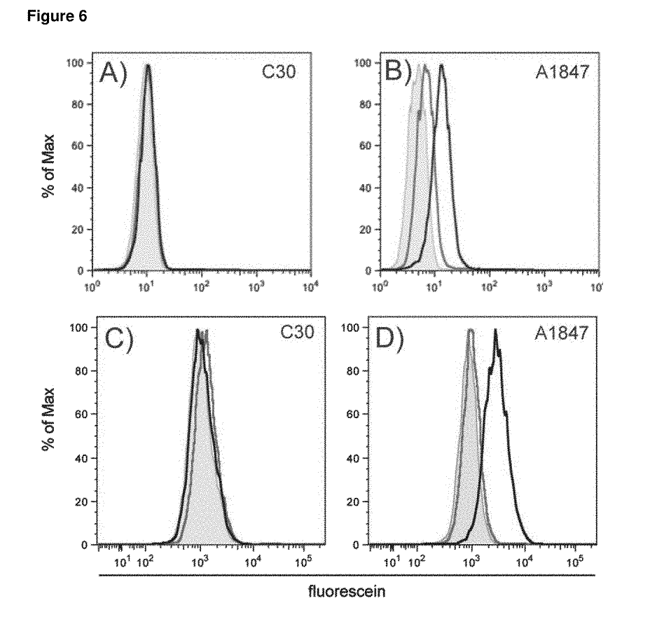

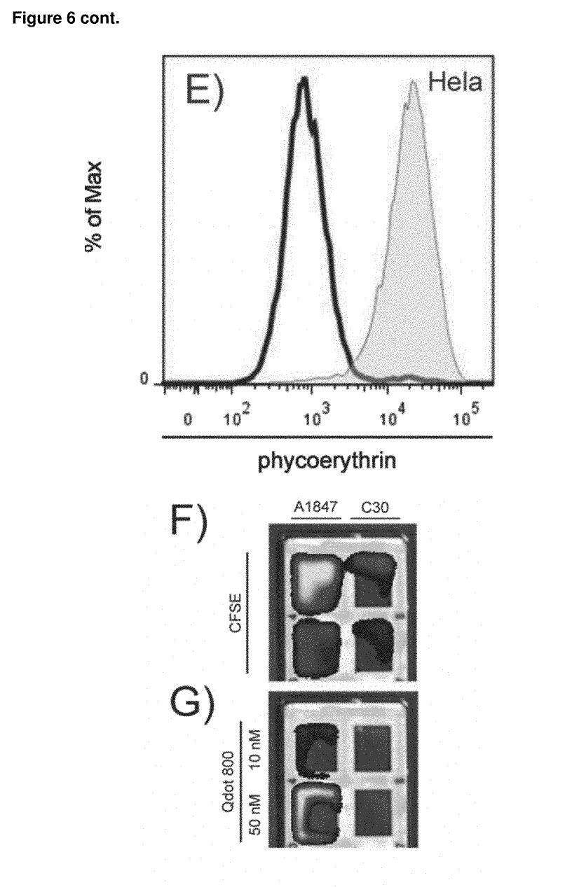

Bb A1 was self-assembled out of the crude yeast culture media onto streptavidin-labeled superparamagnetic iron oxide nanoparticles (SPION) for fluorescent detection. The human ovarian cancer cell line C30 was used as negative control for nonspecific binding evaluation (FIGS. 6A and C). The untargeted nanoparticles and isotype control showed negligible fluorescence over the background cellular autofluorescence, which indicates a low level of nonspecific binding. In contrast, clear fluorescence shifts were observed with Bb A1-functionalized nanoparticles and K1 (black line) on the A1847 human ovarian cancer cell line that overexpresses mesothelin (FIGS. 6B and D). As a negative control, untargeted nanoparticles (gray line) showed fluorescence levels that corresponded to background autofluorescence.

To further demonstrate the versatility of nanobodies as nanoparticle targeting reagents, as an example, Nb-A1 was modified to include a C-terminal cysteine residue (Cys-A1) for site-specific and oriented conjugation to nanoparticles through thiol-maleimide coupling. Adding a free cysteine did not impair nanobody binding to HeLa cells because flow cytometry showed a large fluorescence shift of approximately 1.5 log units (FIG. 6E). Another kind of nanoparticle, fluorescent semiconductor CdSe/CdTe—ZnS nanocrystals quantum dots (QD), were functionalized using Cys-A1 and the binding activity of the resulting fluorescent nanoparticles were further characterized on cells grown in chamber slides. As a control, CFSE was used to directly label cells on slides. Optical imaging at 10 and 50 nM Cys-A1/QD concentrations demonstrated differential binding of the Cys-A1/QD bioconjugates to mesothelin positive (A1847) compared to mesothelin negative (C30) cells (FIGS. 6F and G). These results demonstrate that low concentrations of nanobody-functionalized QD can be used to detect the expression of mesothelin on living cells, without being hindered by non-specific binding to the cell surface.

### EXAMPLE 7

### Nanobody Stability and Targeting at Physiological Temperature

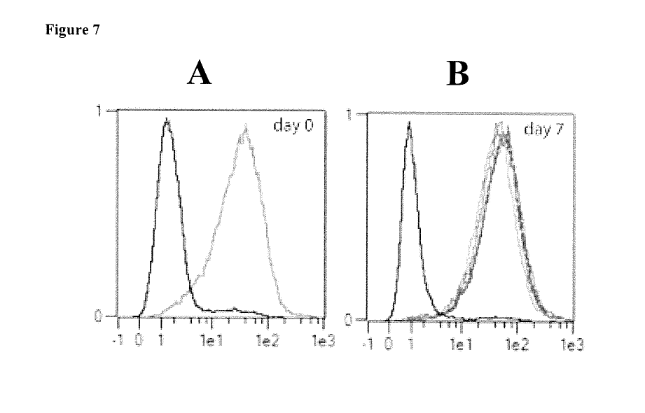

Nanobody-A1 showed similar fluorescence shifts by flow cytometry compared to the initial staining after 7 days at −20, 4, and 37° C. in PBS or after 7 days at 37° C. in 90% human serum (FIG. 7). Accordingly, the nanobodies of the invention and constructs based thereon, e.g., conjugate constructs, exhibit high stability as assessed by standard methods known in the art. For example, the nanobodies and conjugate-constructs disclosed herein exhibit equivalent activity, e.g., binding activity, before and after incubation in PBS or 90% human serum at −20, 4, and 37° C. for 7 days.

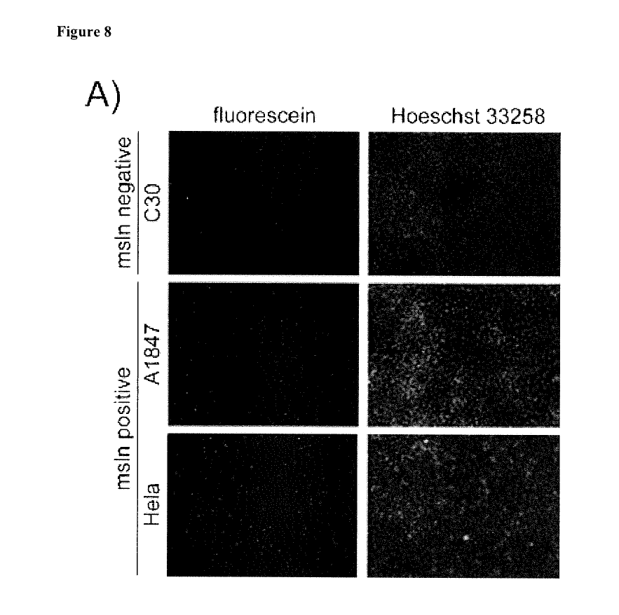

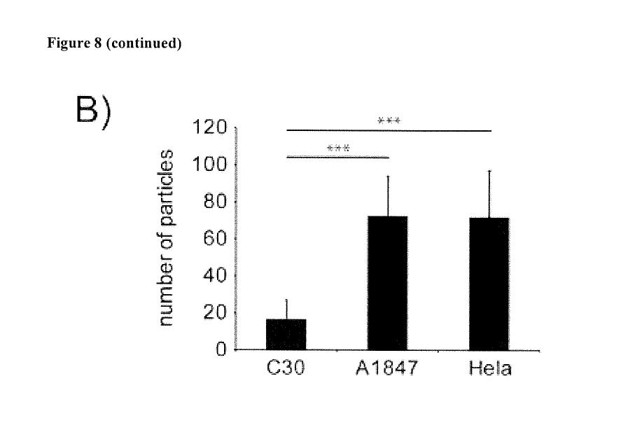

To further validate the potential for Nb-A1 to bind mesothelin in vivo, the nanobody specificity at 37° C. was determined by incubating nanobody-labeled fluorescent compensation beads with mesothelin negative (C30) and mesothelin positive (A1847 and Hela) cells for 4 hr. Fluorescent images showed that Nb-Al was able to discriminate between antigen positive and antigen negative cells at 37° C. (FIG. 8A). Quantitative image analysis using ImageJ determined that the mean number and standard deviation of particles bound to C30, A1847, and Hela cells were 17±10, 73±22, and 72±25, respectively (FIG. 8B).

We claim:

1. An isolated nanobody, or conjugate construct thereof, that binds mesothelin, which nanobody or conjugate-construct, exhibits one or more of the following properties:
(a) binds to mesothelin with a K_{D} of at least 5×10⁻⁸ M or less as determined by surface plasmon resonance analysis;
(b) cross-competes with the nanobody having the amino acid sequence SEQ ID NO:1 or SEQ ID NO:2 for binding to mesothelin; and
(c) cross-competes with the nanobody expressed by a host cell comprising the nucleic acid sequence SEQ ID NO:3 or SEQ ID NO:4 for binding to mesothelin.

2. The isolated nanobody or conjugate-construct according to claim 1 and exhibiting one or both of features (b) and (c), which
(d) cross-competes with the nanobody having the amino acid sequence SEQ ID NO:1 or SEQ ID NO:2 for binding to an epitope of mesothelin; or
(e) cross-competes with the nanobody expressed by a host cell comprising the nucleic acid sequence SEQ ID NO:3 or SEQ ID NO:4 for binding to an epitope of mesothelin.

3. The isolated nanobody or conjugate construct according to claim 1, wherein said mesothelin comprises the amino acid sequence SEQ ID NO:5 or is the mesothelin expressed by a host cell comprising the nucleic acid sequence SEQ ID NO:6.

4. The isolated nanobody or conjugate construct according to claim 2, wherein said nanobody comprises an amino acid sequence that is at least 80% identical to SEQ ID NO:1 or SEQ ID NO:2.

5. The isolated nanobody or conjugate construct according to claim 2, wherein said nanobody comprises one or more of (a) a VHH domain CDR1 comprising the amino acid sequence of SEQ ID NO:7 or SEQ ID NO:10; (b) a VHH domain CDR2 comprising the amino acid sequence of SEQ ID NO:8 or SEQ ID NO:11; and (c) a VHH domain CDR3 comprising the amino acid sequence of SEQ ID NO:9 or SEQ ID NO:12.

6. The isolated nanobody or conjugate-construct according to claim 5, wherein said nanobody comprises (a) a VHH domain CDR1 comprising the amino acid sequence of SEQ ID NO:7; (b) a VHH domain CDR2 comprising the amino acid sequence of SEQ ID NO:8; and (c) a VHH domain CDR3 comprising the amino acid sequence of SEQ ID NO:9.

7. The isolated nanobody or conjugate-construct according to claim 5, wherein said nanobody comprises (a) a VHH domain CDR1 comprising the amino acid sequence of SEQ ID NO:10; (b) a VHH domain CDR2 comprising the amino acid sequence of SEQ ID NO:11; and (c) a VHH domain CDR3 comprising the amino acid sequence of SEQ ID NO:12.

8. An isolated nanobody or isolated conjugate construct comprising the amino acid sequence SEQ ID NO:1 or SEQ ID NO:2.

9. An isolated nanobody or conjugate construct expressed by a host cell comprising the nucleic acid sequence SEQ ID NO:3 or SEQ ID NO:4.

10. The isolated nanobody or conjugate-construct according to claim 9, wherein said host cell is E. coli.

11. An isolated nucleic acid encoding the nanobody or conjugate construct according to claim 1.

12. A host cell comprising the nucleic acid according to claim 11.

13. A method of producing a nanobody or conjugate construct thereof comprising the step of culturing the host cell according to claim 12.

14. The method of claim 13, further comprising recovering the nanobody or conjugate construct from the host cell.

15. A pharmaceutical composition produced by the process of producing a nanobody or conjugate construct thereof comprising the step of culturing the host cell according to claim 12;
recovering the nanobody or conjugate construct produced in the producing step; and
distributing the nanobody or conjugate construct in a pharmaceutically acceptable carrier.

16. A method for the treatment or amelioration of mesothelin-associated cancer, or one or more symptoms thereof, in a subject in need thereof, comprising administering to said subject a therapeutically effective amount of the pharmaceutical composition according to claim 15.

17. A method for detecting mesothelin in a biological sample comprising contacting the sample with a nanobody or conjugate-construct of claim 1 under conditions permissive for binding of said nanobody or conjugate-construct to said mesothelin, and determining whether said nanobody or conjugate-construct binds to said sample.

18. The method according to claim 17, wherein said method is for the diagnosis or confirmation of diagnosis of a mesothelin-associated cancer in a subject, wherein said sample is a sample from said subject, wherein said subject has or is suspected to have a mesothelin-associated cancer, and diagnosing or confirming the diagnosis of said cancer if an increase in binding of the nanobody or conjugate construct to the sample is detected as compared to the binding of the nanobody or conjugate construct to a control sample. The method according to claim 16, wherein the mesothelin-associated cancer is mesothelioma, ovarian cancer, pancreatic cancer or an epithelial tumor.


SEQUENCE LISTING (12 sequences)

SEQ ID NO: 1 - protein, 116 aa, Lama glama
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 2 - protein, 117 aa, Lama glama
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 3 - DNA, 350 bp, Lama glama
caggtgcagctggtgcagtctgggggaggcttggtgcaccctggggggtctctgagactctcctgtgcagcctctggaatcgacctcagtctttatcgcatgcgctggtatcgccaggctccaggaaaggagcgcgacttggtcgcacttataactgatgatggtacttcgtactatgaagactccgtgaagggccgattcaccatcaccagggacaatccctcgaacaaggtgtttctgcaaatgaacagcctgaaacctgaggacacggccgtctattactgtaatgcagagacgcctttatcgccggtcaactactggggccaggggacccaggtcactgtctcctc

SEQ ID NO: 4 - DNA, 351 bp, Lama glama
caggtgcagctggtgcagtctgggggaggattggtgcaggctgggggctctctgagactctcctgtgcaccctctggaagcatcttcggtatccgtaccatggactggtaccgccaggctccagggaaggagcgcgagttggtcgcacgaattacgatggatggtcgggtattccatgcagactccgtgaagggccgattctccggctccagagacggcgcctcgaacgcggtgtatctgcaaatgaacagcctgaaacctgacgacacggccgtctattactgtcgatatagtggcttaacctcaagggaggactactggggcccggggacccaggtcaccgtctcctca

SEQ ID NO: 5 - protein, 285 aa, Homo sapiens [sequence omitted]

SEQ ID NO: 6 - DNA, 855 bp, Homo sapiens
gaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgcttagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggc

SEQ ID NO: 7 - protein, 8 aa, Lama glama
GIDLSLYR

SEQ ID NO: 8 - protein, 7 aa, Lama glama
ITDDGTS

SEQ ID NO: 9 - protein, 11 aa, Lama glama
NAETPLSPVNY

SEQ ID NO: 10 - protein, 8 aa, Lama glama
GSIFGIRT

SEQ ID NO: 11 - protein, 7 aa, Lama glama
ITMDGRV

SEQ ID NO: 12 - protein, 11 aa, Lama glama
RYSGLTSREDY